(19) United States
(12) Patent Application Publication
(10) Pub. No.: US 2025/0137043 A1
(43) Pub. Date: May 1, 2025

(54) METHODS, COMPOSITIONS, AND KITS FOR DETERMINING THE LOCATION OF A NON-CODING RNA IN A BIOLOGICAL SAMPLE

(71) Applicant: 10x Genomics, Inc., Pleasanton, CA (US)

(72) Inventor: Augusto Manuel Tentori, Dublin, CA (US)

(21) Appl. No.: 18/927,479

(22) Filed: Oct. 25, 2024

Related U.S. Application Data
(60) Provisional application No. 63/593,111, filed on Oct. 25, 2023.

(51) Int. Cl. C12Q 1/6841

(52) U.S. Cl. CPC C12Q 1/6841

(57) ABSTRACT

Provided herein are methods, kits, and compositions for determining the spatial location of non-coding RNA, such as siRNA and/or miRNA in a biological sample.

### CROSS-REFERENCE TO RELATED APPLICATIONS

This application claims the benefit of U.S. Provisional Application No. 63/593,111 filed Oct. 25, 2023, which is herein incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Non-coding RNAs such as microRNA (miRNA) and small interfering RNA (siRNA) are small, single-stranded, non-coding RNAs about 20 nucleotides to about 24 nucleotides in length. miRNA and siRNA are involved in post-transcriptional regulation of gene expression and are involved in many biological processes including plant and animal development. It is known that different miRNAs and siRNAs are differentially expressed in various cell types and tissues and methods of identifying the spatial location within a biological sample of such non-coding RNAs are needed.

### SUMMARY

The present disclosure features methods, compositions, and kits for determining the location of non-coding RNAs (e.g., miRNA, siRNA) in a biological sample (e.g., a tissue section). Determining the spatial location and abundance of non-coding RNAs within a biological sample leads to better understanding of spatial heterogeneity in various contexts, including developmental stages of plants and animals. Described herein are methods for capturing non-coding RNAs with nucleic acid probes and capturing said probes via capture domains of capture probes on a substrate. The capture probes also include a spatial barcode which is a unique sequence that identifies a location on the substrate.

Thus provided herein are methods of determining a location of a non-coding RNA in a biological sample, the method including: (a) providing an array including a plurality of capture probes, where a capture probe of the plurality of capture probes includes: (i) a spatial barcode and (ii) a capture domain; (b) contacting the biological sample with a plurality of probes, where a probe of the plurality of probes includes: (i) a nucleic acid sequence capable of hybridizing to at least a portion of the non-coding RNA and (ii) a ligation handle; (c) contacting the biological sample with a plurality of splint oligonucleotides, where a splint oligonucleotide of the plurality of splint oligonucleotides includes: (i) a sequence capable of hybridizing to at least a portion of the ligation handle and (ii) a nucleic acid capture sequence capable of hybridizing to at least a portion of the capture domain of the capture probe; (d) hybridizing the probe to the non-coding RNA and the splint oligonucleotide; (e) ligating the non-coding RNA to the splint oligonucleotide, thereby generating a ligation product; (f) hybridizing the nucleic acid capture sequence of the ligation product to the capture domain of the capture probe; and (g) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

In some embodiments, step (d) occurs prior to step (c) and/or after step (b).

In some embodiments, the non-coding RNA is a siRNA or miRNA, optionally where the miRNA is a pri-miRNA or a pre-miRNA.

In some embodiments, the probe and/or the splint oligonucleotide includes one or more locked nucleic acids.

In some embodiments, the method includes, before or during step (d) the use of one or more minor groove binders, optionally where the one or more minor groove binders includes one or more of duocarmycin A, Chromomycin A3, and alkamin.

In some embodiments, the biological sample is disposed on the array. In some embodiments, the biological sample is disposed on a first substrate and the array is disposed on a second substrate. In some embodiments, the method includes aligning the first substrate with the second substrate such that at least a portion of the biological sample is aligned with at least a portion of the array.

In some embodiments, the method includes releasing or separating the probe from the ligation product, optionally where the releasing includes the use of heat.

In some embodiments, the method includes extending the capture probe using the ligation product as a template, thereby generating an extended capture probe including a sequence complementary to the ligation product; and/or extending the ligation product using the capture probe as a template, thereby generating an extended ligation product including a sequence complementary to the capture probe.

In some embodiments, the nucleic acid capture sequence of the splint oligonucleotide includes a homopolymeric sequence or a fixed sequence. In some embodiments, the homopolymeric sequence is a poly(A) sequence.

In some embodiments, the non-coding RNA is complexed with one or more Argonaute proteins, where the one or more Argonaute proteins includes one or more of: AGO1, AGO2, AGO3, and AGO4, optionally where the one or more Argonaute proteins lacks cleavage activity.

In some embodiments, the method includes imaging the biological sample. In some embodiments, the method includes staining the biological sample, where the staining includes: (i) hematoxylin and/or eosin staining or (ii) a radioisotope, a fluorophore, a chemiluminescent compound, a bioluminescent compound, or a combination thereof.

In some embodiments, the capture probe includes a cleavage domain, one or more functional domains, a unique molecular identifier, or a combination thereof.

In some embodiments, the capture domain of the capture probe includes a fixed sequence or a homopolymeric sequence, optionally where the homopolymeric sequence is a poly(T) sequence.

In some embodiments, the method includes permeabilizing the biological sample, where the permeabilizing includes the use of a protease, optionally where the protease includes pepsin or proteinase K.

In some embodiments, the biological sample is a tissue section, optionally a fresh frozen tissue section or a fixed frozen tissue section.

In some embodiments, the method includes migrating the ligation product to the array or migrating the capture probe from the array to the biological sample, optionally where the capture probe includes a cleavage domain and the method includes cleaving the capture probe at the cleavage domain to release the capture probe from the array.

In some embodiments, the determining includes sequencing; optionally where the method includes sequencing the extended capture probe or a complement thereof or sequencing the extended ligation or a complement thereof.

Also provided herein are methods of determining a location of a non-coding RNA in a tissue sample, the method including: (a) providing an array including a plurality of capture probes, where a capture probe of the plurality of capture probes includes: (i) a spatial barcode and (ii) a capture domain; (b) contacting the tissue sample with a plurality of probes, where a probe of the plurality of probes includes: (i) a nucleic acid sequence capable of hybridizing to at least a portion of the non-coding RNA and (ii) a ligation handle; (c) hybridizing the probe to the non-coding RNA, thereby generating a probe: non-coding RNA complex; (d) contacting the tissue sample with a plurality of splint oligonucleotides, where a splint oligonucleotide of the plurality of splint oligonucleotides includes: (i) a sequence capable of hybridizing to the ligation handle and (ii) a nucleic acid capture sequence capable of hybridizing to at least a portion of the capture domain of the capture probe; (e) hybridizing the splint oligonucleotide to the probe: non-coding RNA complex, where the sequence capable of hybridizing to the ligation handle of the splint oligonucleotide hybridizes to the ligation handle of the probe in the probe: non-coding RNA complex; (f) coupling the non-coding RNA to the splint oligonucleotide, thereby generating a connected probe; (g) hybridizing the connected probe to the capture probe, optionally including hybridizing the nucleic acid capture sequence of the connected probe to the capture domain of the capture probe; and (h) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the tissue sample.

In some embodiments, the coupling in step (f) includes ligation, optionally where the ligation includes chemical ligation or enzymatic ligation, optionally where the enzymatic ligation includes use of a T4 RNA ligase (Rnl2), a PBCV-1 DNA Ligase or Chlorella virus DNA Ligase, a single-stranded DNA ligase, or a T4 DNA ligase.

In some embodiments, the method includes after step (f), releasing or separating the probe from the connected probe, optionally where the releasing or separating includes the use of heat.

In some embodiments, the method includes extending the capture probe using the connected probe as a template, thereby generating an extended capture probe including a sequence complementary to the connected probe; and/or extending the connected probe using the capture probe as a template, thereby generating an extended connected probe including a sequence complementary to the capture probe.

In some embodiments, the determining includes sequencing; optionally where the method includes sequencing the extended capture probe or a complement thereof or sequencing the extended connected probe or a complement thereof.

In some embodiments, a 3′ end of the non-coding RNA is ligated to a 5′ end of the splint oligonucleotide.

In some embodiments, the non-coding RNA is a siRNA or a miRNA, optionally where the miRNA is a pri-miRNA or a pre-miRNA.

In some embodiments, the method includes migrating the connected probe to the array or migrating the capture probe from the array to the biological sample, optionally where the capture probe includes a cleavage domain and the method includes cleaving the capture probe at the cleavage domain to release the capture probe from the array.

In some embodiments, the biological sample is a tissue section, optionally a fresh frozen tissue section or a fixed frozen tissue section.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product.

FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 is a schematic diagram showing an exemplary workflow for spatial capture of non-coding RNA.

FIG. 13 is a schematic diagram showing an exemplary workflow for spatial capture of non-coding RNA.

FIG. 14 is a schematic diagram showing an exemplary workflow for spatial capture of non-coding RNA via in situ reverse transcription.

FIG. 15 is a schematic diagram showing an exemplary workflow for spatial capture of non-coding RNA via a templated ligation reaction.

FIG. 16 is a schematic diagram showing an exemplary workflow for spatial capture of non-coding RNA via a splint oligonucleotide.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9 (10): 5188-96; Kap M. et al., PLOS One.; 6(11): e27704 (2011); and Mathieson W. et al., Am J Clin Pathol.; 146 (1): 25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I) (d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II) (d) (ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

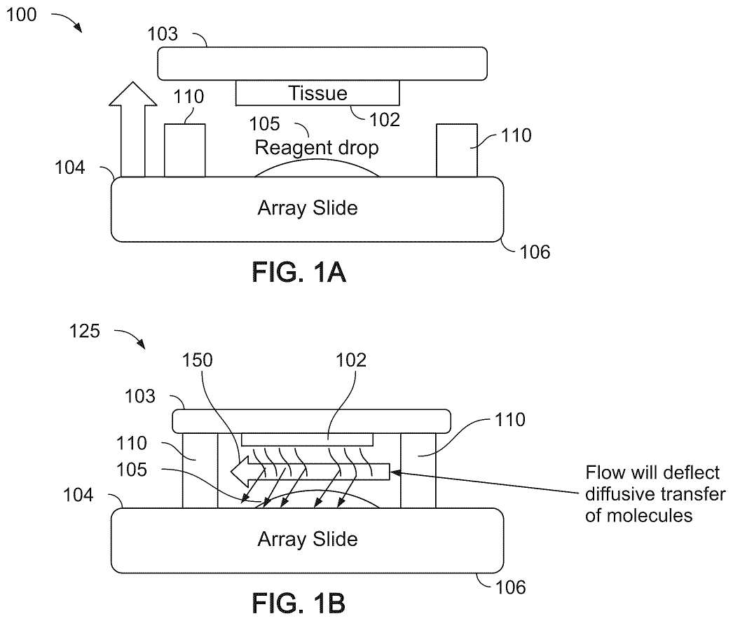

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

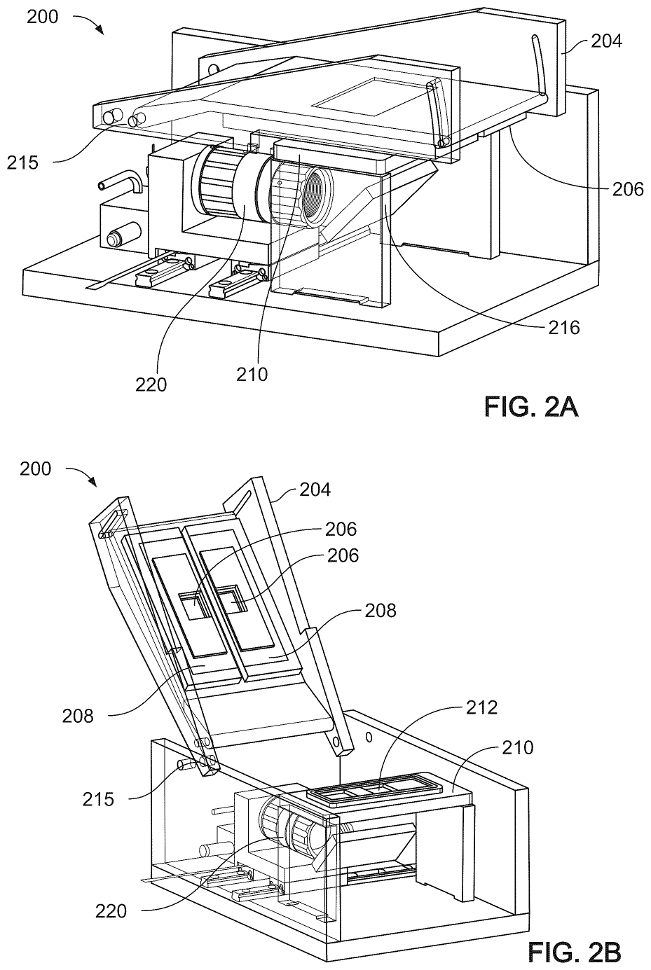

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

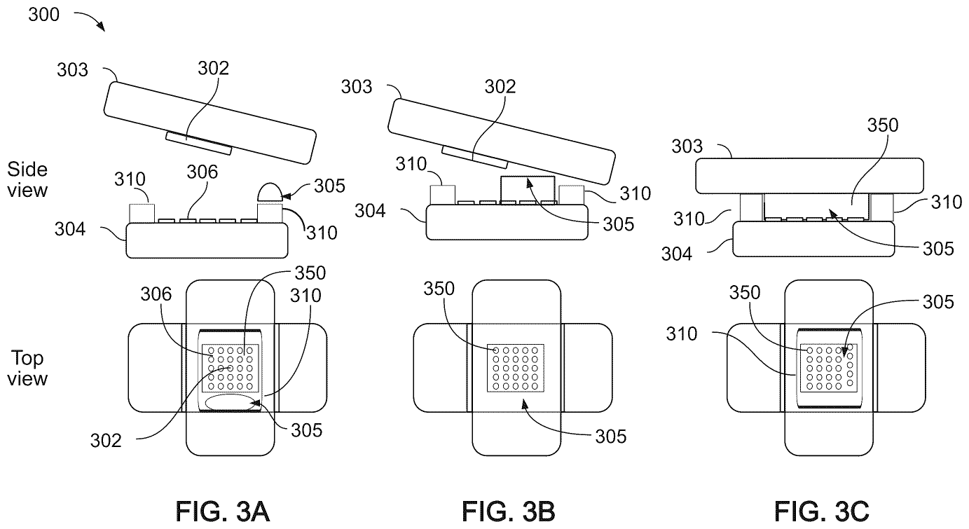

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

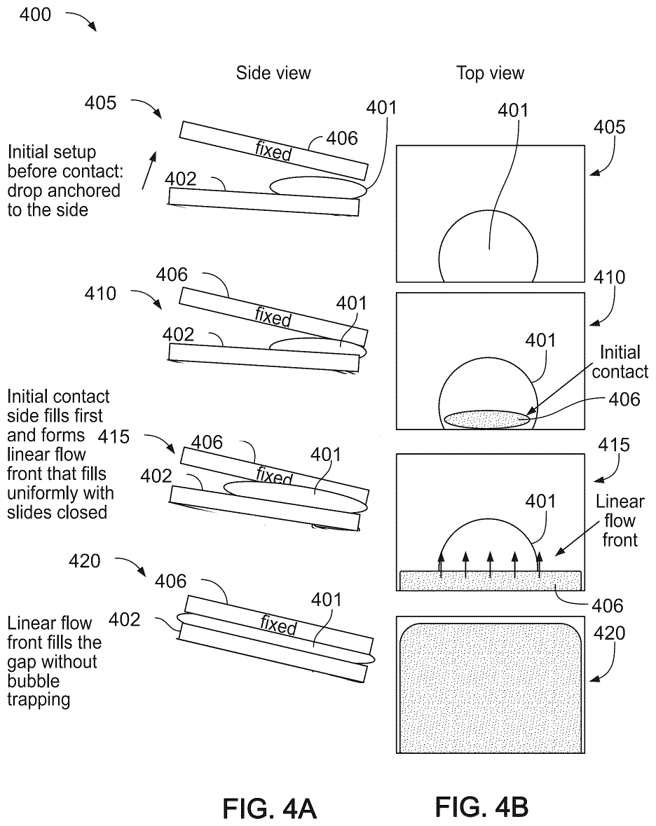

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (e) of PCT Publication No. WO2020/176788 and/or U.S. Patent Application Publication No. 2020/0277663, which is herein incorporated by reference.

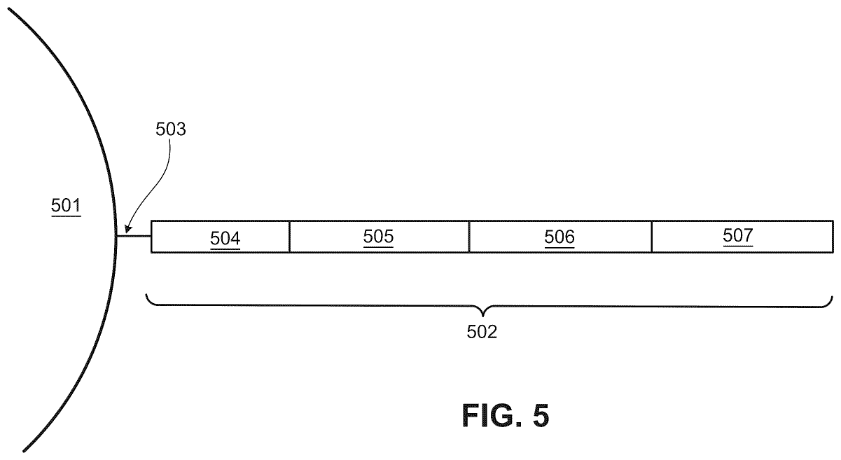

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

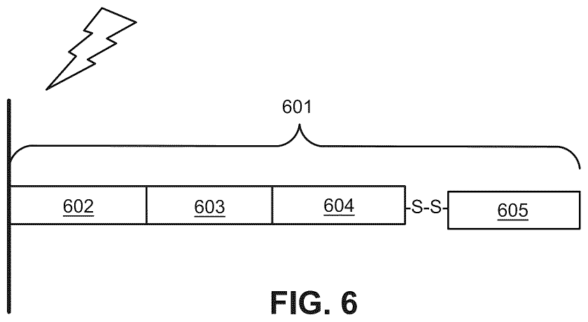

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

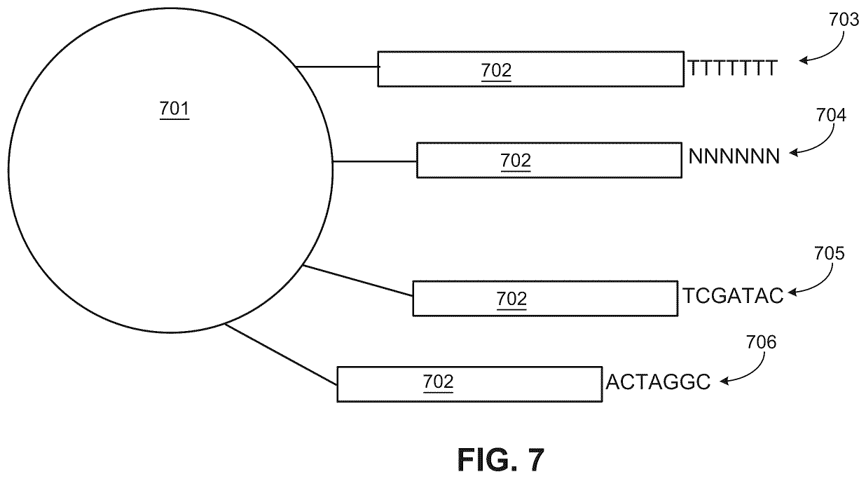

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct.

For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

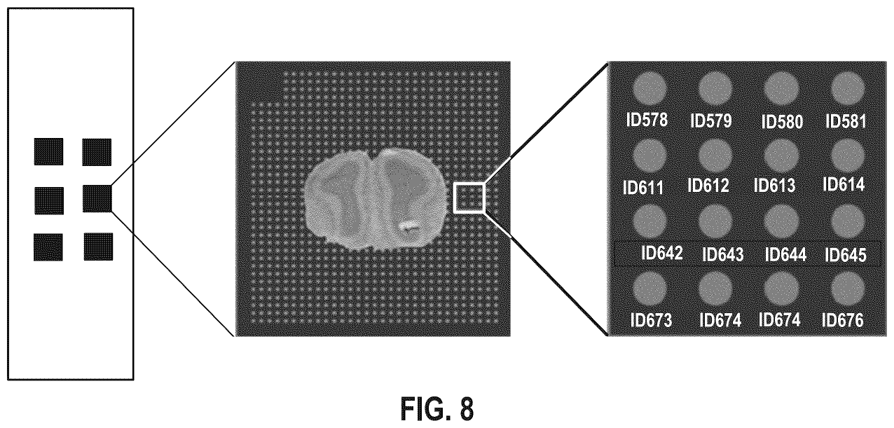

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14): e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

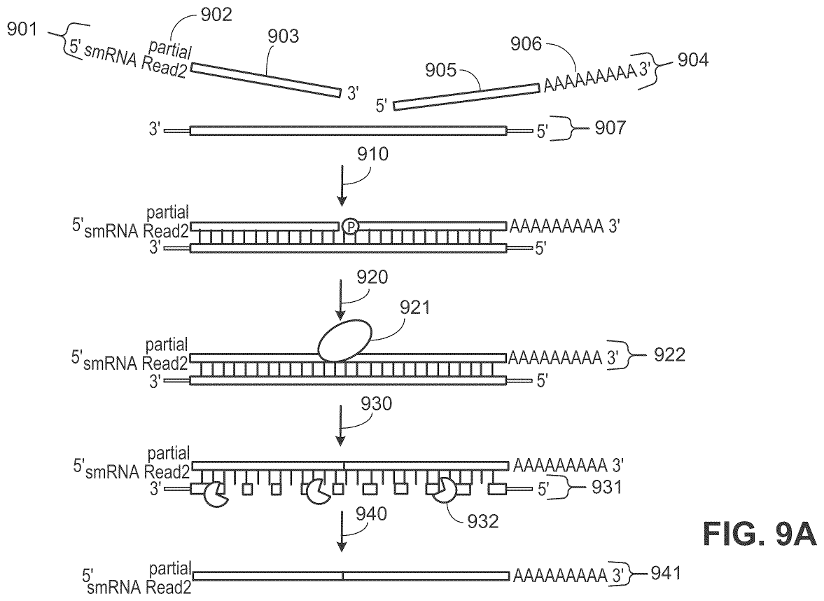

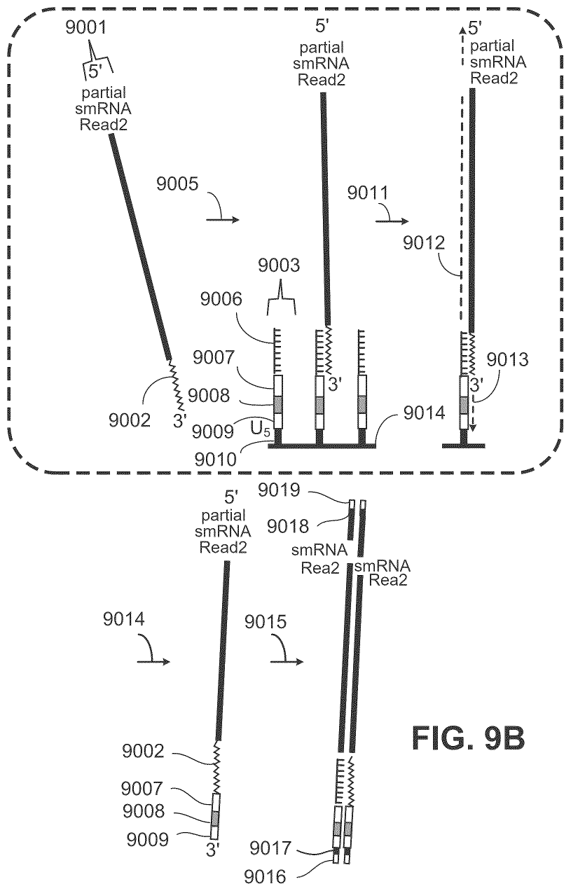

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

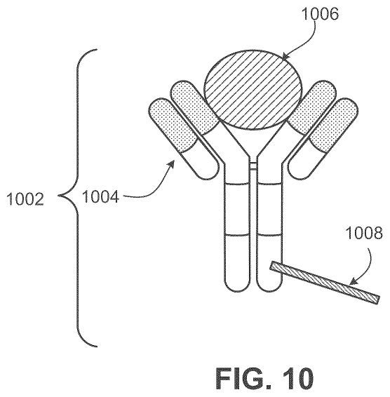

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

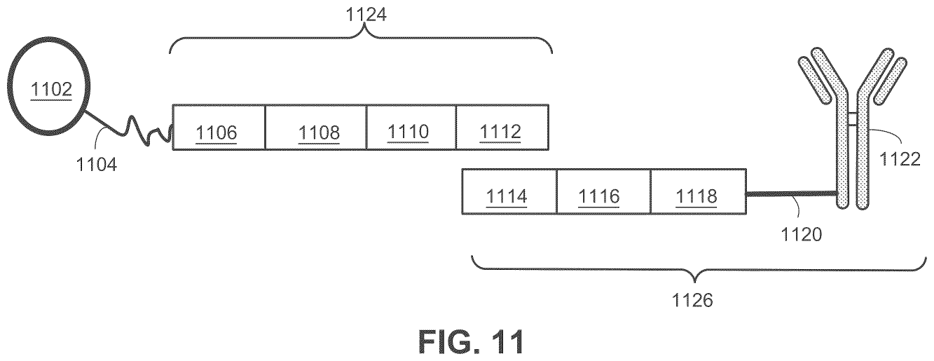

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II) (e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Methods, Compositions, and Kits for Spatial Capture of Non-Coding RNA

### (i) Introduction

Provided herein are methods, kits, and compositions for determining the location and/or abundance of non-coding RNAs (e.g., miRNA, siRNA) in a biological sample. Determining the spatial location and/or abundance of non-coding RNAs within a biological sample leads to better understanding of spatial heterogeneity in various contexts. In general, miRNA and siRNA are involved in post-transcriptional regulation of gene expression through various pathways described herein and are involved in many biological processes including plant and animal development. It is known that different miRNAs and siRNAs are differentially expressed in various cell types and tissues. Additionally, deficiency or excess of non-coding RNAs have been linked to several clinically important diseases. Thus, methods of identifying the spatial location of non-coding RNAs within a biological sample are necessary.

As used herein “non-coding RNA” refers to RNAs that do not encode for a protein product. Non-coding RNAs include small or short interfering RNA (siRNA) and microRNA (miRNA), including processed and/or precursor RNAs of such RNAs. siRNAs are generally about 20 nucleotides to about 24 nucleotides in length. siRNAs generally interfere with expression of specific genes with complementary nucleotide sequences by mediating degradation of the mRNA after transcription. miRNAs are generally about 21 nucleotides to about 23 nucleotides in length. miRNAs generally interfere with expression of specific genes through cleavage of an mRNA, destabilization of an mRNA by shortening its poly(A) tail, and/or inhibiting translation of mRNA into protein. Both miRNAs and siRNAs are single-stranded RNAs found in plants, animals, and some viruses.

As used herein non-coding RNAs also include primary miRNA (pri-miRNA) and/or precursor miRNA (pre-miRNA). As used herein “primary miRNA” or “pri-miRNA” refers to the transcribed nucleic acid (e.g., RNA transcript) from miRNA gene sequences. Pri-miRNAs are then processed into precursor RNAs and miRNAs. Pri-miRNAs are processed via a microprocessor complex comprised of Drosha and DiGeorge Syndrome Critical Region 8 (DGCR8). As used herein “precursor miRNA” or “pre-miRNA” refers to the hairpin precursors of miRNAs formed by the cleavage of pri-miRNA described herein. A comprehensive review of miRNA biogenesis and processing is found in O'Brien, J., et al., Overview of MicroRNA Biogenesis, Mechanisms of Actions, and Circulation, Front. Endocrinology, Vol. 9 (2018), which is incorporated by reference in its entirety.

The pre-miRNAs are exported to the cytoplasm by exportin 5, where they are cleaved by Dicer into short (˜17-25 nucleotide) double-stranded RNA molecules. It is believed that the strand of the pre-miRNA with less 5′ stability then can become bound to the Argonaute protein containing RNA interference silencing complex (RISC) and effect mRNA regulation by binding at 3′ untranslated region (3′ UTR) of mRNA having homology to the miRNA (target mRNA) or by directing transport of the mRNA into bodies. Binding results in either cleavage of the target mRNA if there is 100% complementarity between the miRNA and the target RNA (RNA interference) or down-regulation of expression (without cleavage) by binding to the target mRNA and blocking translation or directing mRNA decay. A useful resource for miRNA information is available from the Sanger Institute, which maintains a registry of miRNA at http:/microrna.sanger.ac.uk/sequences/. The miRBase Sequence database includes the nucleotide sequences and annotations of published miRNA from a variety of sources. The miRBase Registry provides unique names for novel miRNA genes that comply with conventional naming nomenclature for new miRNA prior to publication. The miRBase Targets is a resource for predicated miRNA targets in animals. The databases are updated frequently and thus provide a comprehensive source of useful miRNA nucleotide sequences.

In some instances, the methods descried herein include aligning (e.g., sandwiching) a first substrate having the biological sample with a second substrate that includes an array having a plurality of capture probes, thereby “sandwiching” the biological sample between the two substrates. As used herein, “aligning” encompasses bringing a first side of the second substrate comprising the array into contact or proximity with a first side of the first substrate comprising the biological sample, where the first side of the first substrate and the first side of the second substrate are positioned opposite each other. See, for example, FIGS. 1-4. Upon bringing the biological sample and the substrate having a plurality of capture probes into proximity, the location and/or abundance of non-coding RNAs in the biological sample can be determined, as provided herein. The sandwiching embodiment of the disclosed methods provide an advantage in that, prior to products derived from non-coding RNA capture by the capture probe, most-if not all-steps can be performed on a substrate that does not have capture probes, thereby providing a method that is cost effective.

The methods disclosed herein can also be performed on a single substrate, wherein the single substrate comprises the plurality of capture probes, each having a capture domain and spatial barcode. In these instances, all steps of products derived from non-coding RNA capture occur on the single substrate (i.e., there is no transfer of an analyte or analyte-derived molecule from one substrate to a second substrate). For example, the biological sample can be disposed on the single substrate comprising the plurality of capture probes.

In both setups (sandwiching or single substrate), after products derived from non-coding RNA capture, the capture probe can be extended and/or the product derived from the non-coding RNA can be extended; either extended product can be optionally amplified, purified, and/or sequenced in order to determine the abundance and/or location of the non-coding RNA in the biological sample.

It is appreciated that the methods, compositions, and kits provided herein can be applied to products derived from non-coding RNA. As used herein, a product derived from non-coding RNA includes, without limitation a ligation product from a templated ligation assay, a product of reverse transcription (e.g., an extended capture probe), a probe, and a non-coding RNA barcode (e.g., a non-coding RNA barcode that identifies that non-coding RNA). In some embodiments, the products derived from non-coding RNA comprise RNA and/or DNA.

In some instances, the methods, kits, and compositions disclosed herein provide efficient release of an analyte or products derived from non-coding RNA from a biological sample so that it can be easily captured or detected using methods disclosed herein.

In some instances, the methods, kits, and compositions disclosed herein allow for detection of products derived from non-coding RNA from different biological samples using a single array comprising a plurality of capture probes. As such, in some instances, the methods, kits, and compositions allow for serial capture of analytes or products derived from non-coding RNA from multiple samples. The products derived from non-coding RNA can then be demultiplexed using biological-sample-specific index sequences to identify its biological sample origin.

Embodiments of the methods, kits, and compositions disclosed herein are provided below.

### Exemplary First and Second Substrates

In some instances, the biological sample is placed (e.g., mounted or otherwise immobilized) on a first substrate. The first substrate can be any solid or semi-solid support upon which a biological sample can be mounted. In some instances, the first substrate is a slide. In some instances, the slide is a glass slide. In some embodiments, the substrate is made of glass, silicon, paper, hydrogel, polymer monoliths, or other material known in the art. In some embodiments, the first substrate is comprised of an inert material or matrix (e.g., glass slides) that has been functionalized by, for example, treating the substrate with a material comprising reactive groups which facilitate mounting of the biological sample.

In some embodiments, the first substrate does not comprise a plurality (e.g., array) of capture probes, each comprising a spatial barcode.

A substrate, e.g., a first substrate and/or a second substrate, can generally have any suitable form or format. For example, a substrate can be flat, curved, e.g., convexly or concavely curved. For example, a first substrate can be curved towards the area where the interaction between a biological sample, e.g., tissue sample, and a first substrate takes place. In some embodiments, a substrate is flat, e.g., planar, chip, or slide. A substrate can contain one or more patterned surfaces within the first substrate (e.g., channels, wells, projections, ridges, divots, etc.).

A substrate, e.g., a first substrate and/or second substrate, can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments wherein a substrate structure is flat, the substrate structure can be any appropriate type of support having a flat surface (e.g., a chip or a slide such as a microscope slide).

First and/or second substrates can optionally include various structures such as, but not limited to, projections, ridges, and channels. A substrate can be micropatterned to limit lateral diffusion of analytes (e.g., to improve resolution of the spatial analysis). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a first and/or second substrate is modified to contain one or more wells, using techniques such as (but not limited to) stamping, microetching, or molding techniques. In some embodiments in which a first and/or second substrate includes one or more wells, the first substrate can be a concavity slide or cavity slide. For example, wells can be formed by one or more shallow depressions on the surface of the first and/or second substrate. In some embodiments, where a first and/or second substrate includes one or more wells, the wells can be formed by attaching a cassette (e.g., a cassette containing one or more chambers) to a surface of the first substrate structure.

In some embodiments where the first and/or second substrate is modified to contain one or more structures, including but not limited to, wells, projections, ridges, features, or markings, the structures can include physically altered sites. For example, a first and/or second substrate modified with various structures can include physical properties, including, but not limited to, physical configurations, magnetic or compressive forces, chemically functionalized sites, chemically altered sites, and/or electrostatically altered sites. In some embodiments where the first substrate is modified to contain various structures, including but not limited to wells, projections, ridges, features, or markings, the structures are applied in a pattern. Alternatively, the structures can be randomly distributed.

In some embodiments, a first substrate includes one or more markings on its surface, e.g., to provide guidance for aligning at least a portion of the biological sample with a plurality of capture probes on the second substrate during a sandwich process disclosed herein. For example, the first substrate can include a sample area indicator identifying the sample area. In some embodiments, during a sandwiching process described herein the sample area indicator on the first substrate is aligned with an area of the second substrate comprising a plurality of capture probes. In some embodiments, the first and/or second substrate can include a fiducial marker. In some embodiments, the first and/or second substrate does not comprise a fiducial marker. In some embodiments, the first substrate does not comprise a fiducial marker and the second substrate comprises a fiducial marker. Such markings can be made using techniques including, but not limited to, printing, sand-blasting, and depositing on the surface.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, and colorimetric labels. The use of fiducial markers to stabilize and orient biological samples is described, for example, in Carter et al., Applied Optics 46:421-427, 2007), the entire contents of which are incorporated herein by reference. In some embodiments, a fiducial marker can be a physical particle (e.g., a nanoparticle, a microsphere, a nanosphere, a bead, a post, or any of the other exemplary physical particles described herein or known in the art).

In some embodiments, a fiducial marker can be present on a first substrate to provide orientation of the biological sample. In some embodiments, a microsphere can be coupled to a first substrate to aid in orientation of the biological sample. In some examples, a microsphere coupled to a first substrate can produce an optical signal (e.g., fluorescence). In some embodiments, a quantum dot can be coupled to the first substrate to aid in the orientation of the biological sample. In some examples, a quantum dot coupled to a first substrate can produce an optical signal.

In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Although not required, it can be advantageous to use a marker that can be detected using the same conditions (e.g., imaging conditions) used to detect a labelled cDNA.

In some embodiments, a fiducial marker can be randomly placed in the field of view. For example, an oligonucleotide containing a fluorophore can be randomly printed, stamped, synthesized, or attached to a first substrate (e.g., a glass slide) at a random position on the first substrate. A tissue section can be contacted with the first substrate such that the oligonucleotide containing the fluorophore contacts, or is in proximity to, a cell from the tissue section or a component of the cell (e.g., an mRNA or DNA molecule). An image of the first substrate and the tissue section can be obtained, and the position of the fluorophore within the tissue section image can be determined (e.g., by reviewing an optical image of the tissue section overlaid with the fluorophore detection). In some embodiments, fiducial markers can be precisely placed in the field of view (e.g., at known locations on a first substrate). In this instance, a fiducial marker can be stamped, attached, or synthesized on the first substrate and contacted with a biological sample. Typically, an image of the sample and the fiducial marker is taken, and the position of the fiducial marker on the first substrate can be confirmed by viewing the image.

In some embodiments, a fiducial marker can be an immobilized molecule (e.g., a physical particle) attached to the first substrate. For example, a fiducial marker can be a nanoparticle, e.g., a nanorod, a nanowire, a nanocube, a nanopyramid, or a spherical nanoparticle. In some examples, the nanoparticle can be made of a heavy metal (e.g., gold). In some embodiments, the nanoparticle can be made from diamond. In some embodiments, the fiducial marker can be visible by eye.

A wide variety of different first substrates can be used for the foregoing purposes. In general, a first substrate can be any suitable support material. Exemplary first substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Among the examples of first substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In another example, a first substrate can be a flow cell. Flow cells can be formed of any of the foregoing materials, and can include channels that permit reagents, solvents, features, and analytes to pass through the flow cell. In some embodiments, a hydrogel embedded biological sample is assembled in a flow cell (e.g., the flow cell is utilized to introduce the hydrogel to the biological sample). In some embodiments, a hydrogel embedded biological sample is not assembled in a flow cell. In some embodiments, the hydrogel embedded biological sample can then be prepared and/or isometrically expanded as described herein.

Exemplary substrates similar to the first substrate (e.g., a substrate having no capture probes) and/or the second substrate are described in Section (I) above and in WO 2020/123320, which is hereby incorporated by reference in its entirety.

### (ii) Capture of Non-Coding RNA

Provided herein are methods of determining a location of a non-coding RNA in a biological sample, the method including: (a) providing an array including a plurality of capture probes, where a capture probe of the plurality of capture probes includes: (i) a spatial barcode and (ii) a capture domain; (b) contacting the biological sample with a probe, where the probe includes: (i) a ncRNA-binding domain comprising a nucleic acid sequence capable of hybridizing to at least a portion of the non-coding RNA and (ii) a nucleic acid capture sequence capable of hybridizing to at least a portion of the capture domain; (c) hybridizing the ncRNA-binding domain of the probe to the non-coding RNA; (d) hybridizing the nucleic acid capture sequence of (ii) the probe to the capture domain of the capture probe; and (e) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

In some embodiments, the method includes releasing the non-coding RNA from the probe after step (c). In some embodiments, the releasing includes the use of an RNase. In some embodiments, the RNase is one or more of RNase A, RNase P, RNase T1, or RNase H. An endoribonuclease such as RNase H specifically cleaves RNA in RNA: DNA hybrids. In some embodiments, the RNase H is RNase H1 or RNase H2. In some embodiments, the releasing further includes the use of a protease. For example, the non-coding RNA might be complexed with a protein (e.g., an Ago1-4 protein) and the protease may degrade the protein to release the non-coding RNA. In some embodiments, the protease is Proteinase K.

In some embodiments, the probe includes a nucleic acid sequence including a non-coding RNA barcode disposed between the ncRNA-binding domain of the probe and the nucleic acid capture sequence of the probe. In some embodiments, the nucleic acid sequence of the non-coding RNA barcode identifies the non-coding RNA hybridized to the probe.

In some embodiments, the method includes extending the capture probe using the probe as an extension template after step (d), thereby generating an extended capture probe. In some embodiments, the extending includes the use of a polymerase.

In some embodiments, the method includes the use of a template switch oligonucleotide after step (d). In some embodiments, the method includes extending the probe using the capture probe as a template after step (d), thereby generating a sequence complementary to the extended capture probe.

Also provided herein are methods of determining a location of a non-coding RNA in a biological sample, the method including: (a) providing an array including a plurality of capture probes, where a capture probe of the plurality of capture probes includes: (i) a spatial barcode and (ii) a capture domain; (b) contacting the biological sample with a probe, where the probe includes a ncRNA-binding domain comprising a nucleic acid sequence capable of hybridizing to at least a portion of the non-coding RNA; (c) hybridizing the probe to the non-coding RNA and extending the probe using the non-coding RNA as an extension template, thereby generating an extended probe; (d) incorporating a polynucleotide sequence including at least three nucleotides to the 3′ end of the extended probe; (e) hybridizing an adaptor to the polynucleotide sequence, where the adaptor includes: (i) a nucleic acid sequence that hybridizes to the polynucleotide sequence and (ii) a nucleic acid sequence complementary to a nucleic acid capture sequence, where the nucleic acid capture sequence is capable of hybridizing to at least a portion of the capture domain; (f) extending the extended probe using the adaptor as an extension template, thereby incorporating the nucleic acid capture sequence into the extended probe; (g) hybridizing the nucleic acid capture sequence of the extended probe to the capture domain; and (h) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

In some embodiments, the method includes after step (f) releasing the non-coding RNA from the extended probe. In some embodiments, the releasing includes the use of an exonuclease. In some embodiments, the exonuclease is a T7 exonuclease.

In some embodiments, the adaptor includes a template switch oligonucleotide. In some embodiments, 5′ end of the template switch oligonucleotide is phosphorylated. In some embodiments, the exonuclease is lambda exonuclease.

In some embodiments, the method includes after step (g), extending the capture probe using the extended probe as a template, thereby generating an extended capture probe. In some embodiments, the extending includes the use of a polymerase. In some embodiments, the polymerase is a DNA polymerase. In some embodiments, the method includes generating a second strand complementary to the extended capture probe.

The polynucleotide sequence incorporated at the end of the cDNA (e.g., reverse transcribed non-coding RNA) can be a heteropolynucleotide sequence or a homopolynucleotide sequence. For example, a reverse transcriptase or a terminal transferase can add a polynucleotide sequence in a template-independent manner (e.g., at least three non-templated nucleotides). In some embodiments, the polynucleotide sequence is a heteropolynucleotide sequence (e.g., CGC). In some embodiments, the polynucleotide sequence is a homopolynucleotide sequence (e.g., CCC). The adaptor that includes a complement of the polynucleotide sequence and a complement of the capture sequence can be hybridized to the polynucleotide sequence incorporated at the end (e.g., 3′ end) of the cDNA. The 3′ end of the cDNA is extended using the adaptor as a template (resulting in a non-coding RNA/DNA (e.g., non-coding RNA/cDNA) duplex). The non-coding RNA can be removed (e.g., via digestion, denaturation, etc.) resulting in a single-stranded product that serves as a product derived from the non-coding RNA which can be captured by a capture probe on a spatial array.

In some embodiments, the methods, kits, and compositions described herein utilize templated ligation to detect the non-coding RNA. As used herein, spatial “templated ligation,” or is a process wherein individual probes (e.g., a first probe, a second probe) in a probe pair hybridize to adjacent sequences of non-coding RNA in a biological sample (e.g., a tissue section). In some examples, the probe pair hybridize to non-adjacent sequences a gap-filling reaction is performed. The probes are then coupled (e.g., ligated) together, thereby creating a ligation product. Templated ligation is disclosed in PCT Publ. No. WO 2021/133849 and US Publ. No. US 2021/0285046 A1, each of which is incorporated by reference in its entirety.

An advantage to templated ligation is that it allows for enhanced specificity in detection of non-coding RNA (e.g., low expressing analytes) because both probes must hybridize to the non-coding RNA in order for the coupling (e.g., ligating) reaction to occur. As used herein, “coupling” refers to an interaction between two probes that results in a single ligation product that comprises the two probes. In some instances, coupling is achieved through ligation. In some instances, coupling is achieved through extension of one probe to the second probe followed by ligation.

The ligation product that results from the coupling (e.g., ligation) of the two probes can serve as a proxy for the non-coding RNA. Further, it is appreciated that probe pairs can be designed to cover any non-coding RNA gene of interest.

Thus, also provided herein are methods of determining a location of a non-coding RNA in a biological sample, the method including: (a) providing an array including a plurality of capture probes, where a capture probe of the plurality of capture probes includes: (i) a spatial barcode and (ii) a capture domain; (b) contacting the biological sample with a plurality of first probes and a plurality of second probes, where the plurality of first probes and the plurality of second probes target a plurality of non-coding RNAs in the biological sample, and where a first probe of the plurality of first probes and a second probe of the plurality of second probes include sequences that are capable of hybridizing to the non-coding RNA, and where the second probe includes a nucleic acid capture sequence that is capable of hybridizing to at least a portion of the capture domain of the capture probe; (c) hybridizing the first probe and the second probe to the non-coding RNA; (d) ligating the first probe and the second probe, thereby generating a ligation product; (e) hybridizing the nucleic acid capture sequence of the ligation product to the capture domain of the capture probe; and (f) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

In some embodiments, the first probe and/or the second probe include one or more locked nucleic acids.

In some embodiments, the method includes after step (d) releasing the non-coding RNA from the ligation product. In some embodiments, the releasing includes the use of an RNase. In some embodiments, the RNase is one or more of RNase A, RNase P, RNase T1, or RNase H. In some embodiments, the releasing further includes the use of a protease (e.g., Proteinase K).

In some embodiments, the nucleic acid capture sequence includes a homopolymeric sequence or a fixed sequence. As used herein, a “fixed sequence” is a non-random sequence. For example, it is only necessary for the capture sequence and the capture domain to be substantially complementary to each other, such that the capture domain and/or the capture sequence including a fixed sequence can hybridize to one another.

In some embodiments, the homopolymeric sequence is a poly(A) sequence.

In some embodiments, the method includes extending the capture probe using the ligation product as a template, thereby generating an extended capture probe. In some embodiments, the extending includes the use of a polymerase. In some embodiments, the method includes extending the ligation product using the extended capture probe as a template, thereby generating an extended ligation product complementary to the extended capture probe.

In some embodiments, the methods as disclosed herein include hybridizing of one or more probe pairs to adjacent or nearby sequences of a non-coding RNA. In some embodiments, the probe pairs include sequences that are complementary or substantially complementary to a non-coding RNA. For example, in some embodiments, each probe includes a sequence that is complementary or substantially complementary to a non-coding RNA of interest. In some embodiments, each non-coding RNA includes a first target region and a second target region. In some embodiments, the methods include providing a plurality of first probes and a plurality of second probes, where a pair of probes for a non-coding RNA comprises both a first and second probe. In some embodiments, a first probe hybridizes to a first target region of the non-coding RNA, and the second probe hybridizes to a second, adjacent or nearly adjacent target region of the non-coding RNA.

In some instances, the probes are DNA molecules. In some instances, the first probe is a DNA molecule. In some instances, the second probe is a DNA molecule. In some instances, the first probe comprises at least two ribonucleic acid bases at the 3′ end. In some instances, the second probe comprises a phosphorylated nucleotide at the 5′ end.

Probe pairs can be designed using methods known in the art. In some instances, the methods disclosed herein utilize about 500, about 1000, about 2000, about 3000, about 4000, about 5000, or more probe pairs.

In some embodiments, one of the probes of the pair of probes for templated ligation includes a poly(A) sequence or a complement thereof. In some instances, the poly(A) sequence or a complement thereof is on 5′ end of one of the probes. In some instances, the poly(A) sequence or a complement thereof is on 3′ end of one of the probes. In some embodiments, one probe of the pair of probes for RTL includes a degenerate or UMI sequence. In some embodiments, the UMI sequence is specific to a particular target or set of targets. In some instances, the UMI sequence or a complement thereof is on 5′ end of one of the probes. In some instances, the UMI sequence or a complement thereof is on 3′ end of one of the probes.

In some instances, the first and second target regions of the non-coding RNA are directly adjacent to one another. In some embodiments, the complementary sequences to which the first probe and the second probe hybridize are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, or about 150 nucleotides away from each other. Gaps between the probes can be first be filled prior to coupling (e.g., ligation), using, for example, dNTPs in combination with a polymerase such as polymerase mu, DNA polymerase, RNA polymerase, reverse transcriptase, VENT polymerase, Taq polymerase, and/or any combinations, derivatives, and variants (e.g., engineered mutants) thereof. In some embodiments, when the first and second probes are separated from each other by one or more nucleotides, deoxyribonucleotides are used to extend and couple (e.g., ligate) the first and second probes.

In some embodiments, the first and/or second probe as disclosed herein includes at least two ribonucleic acid bases at the 3′ end; a functional sequence; a phosphorylated nucleotide at the 5′ end; and/or a capture probe binding domain. In some embodiments, the functional sequence is a primer sequence.

The “capture sequence” is a sequence that is complementary to a capture domain present in a capture probe. In some embodiments, the capture sequence includes a poly(A) sequence. In some embodiments, the capture sequence includes a poly-uridine sequence, a poly-thymidine sequence, or a combination thereof. In some embodiments, the capture sequence includes a random sequence (e.g., a random hexamer or octamer). In some embodiments, a capture probe binding domain blocking moiety that interacts with the capture probe binding domain is provided. In some embodiments, a capture sequence blocking moiety includes a sequence that is complementary or substantially complementary to a capture sequence. In some embodiments, a capture sequence blocking moiety prevents the capture sequence from binding the capture probe when present. In some embodiments, a capture sequence blocking moiety is removed prior to binding the capture sequence (e.g., present in a ligation product) to a capture probe. In some embodiments, a capture sequence blocking moiety comprises a poly-uridine sequence, a poly-thymidine sequence, or a combination thereof.

Hybridization of the probes (e.g., any of the probes described herein) to the non-coding RNA can occur at a target region having a sequence that is 100% complementary to the probe(s). In some embodiments, hybridization can occur at a target region having a sequence that is at least (e.g. at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe(s).

In some embodiments, methods disclosed herein include a wash step after hybridizing probes to non-coding RNAs. The wash step removes any unbound oligonucleotide probes and can be performed using any technique known in the art. In some embodiments, a pre-hybridization buffer is used to wash the sample. In some embodiments, a phosphate buffer is used. In some embodiments, multiple wash steps are performed to remove unbound oligonucleotides. For example, it is advantageous to decrease the amount of unhybridized probes present in a biological sample as they may interfere with downstream applications and methods.

In some embodiments, the first probe and the second probes are on a contiguous nucleic acid sequence. In some embodiments, the first probe is on 3′ end of the contiguous nucleic acid sequence. In some embodiments, the first probe is on 5′ end of the contiguous nucleic acid sequence. In some embodiments, the second probe is on 3′ end of the contiguous nucleic acid sequence. In some embodiments, the second probe is on 5′ end of the contiguous nucleic acid sequence.

Also provided herein are methods of determining a location of a non-coding RNA in a biological sample, the method including: (a) providing an array including a plurality of capture probes, where a capture probe of the plurality of capture probes includes: (i) a spatial barcode and (ii) a capture domain; (b) contacting the biological sample with a plurality of probes, where a probe of the plurality of probes includes: (i) a nucleic acid sequence capable of hybridizing to at least a portion of the non-coding RNA and (ii) a ligation handle; (c) contacting the biological sample with a plurality of splint oligonucleotides, where a splint oligonucleotide of the plurality of splint oligonucleotides includes: (i) a sequence capable of hybridizing to at least a portion of the ligation handle and (ii) a nucleic acid capture sequence capable of hybridizing to at least a portion of the capture domain of the capture probe; (d) hybridizing the probe to the non-coding RNA and the splint oligonucleotide; (e) ligating the non-coding RNA to the splint oligonucleotide, thereby generating a ligation product; (f) hybridizing the nucleic acid capture sequence of the ligation product to the capture domain of the capture probe; and (g) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

Also provided herein are methods of determining a location of a non-coding RNA in a tissue sample, the method including: (a) providing an array including a plurality of capture probes, where a capture probe of the plurality of capture probes includes: (i) a spatial barcode and (ii) a capture domain; (b) contacting the tissue sample with a plurality of probes, where a probe of the plurality of probes includes: (i) a nucleic acid sequence capable of hybridizing to at least a portion of the non-coding RNA and (ii) a ligation handle; (c) hybridizing the probe to the non-coding RNA, thereby generating a probe: non-coding RNA complex; (d) contacting the tissue sample with a plurality of splint oligonucleotides, where a splint oligonucleotide of the plurality of splint oligonucleotides includes: (i) a sequence capable of hybridizing to the ligation handle and (ii) a nucleic acid capture sequence capable of hybridizing to at least a portion of the capture domain of the capture probe; (e) hybridizing the splint oligonucleotide to the probe: non-coding RNA complex, where the sequence capable of hybridizing to the ligation handle of the splint oligonucleotide hybridizes to the ligation handle of the probe in the probe: non-coding RNA complex; (f) coupling the non-coding RNA to the splint oligonucleotide, thereby generating a connected probe; (g) hybridizing the connected probe to the capture probe, optionally including hybridizing the nucleic acid capture sequence of the connected probe to the capture domain of the capture probe; and (h) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the tissue sample.

In some embodiments, a method of determining a location of a non-coding RNA in a tissue sample includes: (a) providing an array comprising a plurality of capture probes, wherein a capture probe of the plurality of capture probes comprises: (i) a spatial barcode and (ii) a capture domain; (b) contacting the tissue sample with a plurality of probes, wherein a probe of the plurality of probes comprises: (i) a nucleic acid sequence capable of hybridizing to at least a portion of the non-coding RNA and (ii) a ligation handle; (c) hybridizing the probe to the non-coding RNA, thereby generating a probe: non-coding RNA complex; (d) contacting the tissue sample with a plurality of splint oligonucleotides, wherein a splint oligonucleotide of the plurality of splint oligonucleotides comprises: (i) a sequence capable of hybridizing to the ligation handle and (ii) a nucleic acid capture sequence capable of hybridizing to at least a portion of the capture domain of the capture probe; (e) hybridizing the splint oligonucleotide to the probe: non-coding RNA complex, wherein the sequence capable of hybridizing to the ligation handle of the splint oligonucleotide hybridizes to the ligation handle of the probe in the probe: non-coding RNA complex; (f) coupling the non-coding RNA to the splint oligonucleotide, thereby generating a connected probe; (g) hybridizing the connected probe to the capture probe, optionally comprising hybridizing the nucleic acid capture sequence of the connected probe to the capture domain of the capture probe; and (h) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the tissue sample.

In some embodiments, the coupling includes ligation. The ligation can include chemical ligation or enzymatic ligation. For example, the enzymatic ligation can include use of a T4 RNA ligase (Rnl2), a PBCV-1 DNA Ligase or Chlorella virus DNA Ligase, a single-stranded DNA ligase, or a T4 DNA ligase.

In some embodiments of the disclosed methods, a 3′ end of the non-coding RNA is ligated to a 5′ end of the splint oligonucleotide. In some embodiments, the probe and/or the splint oligonucleotide includes one or more locked nucleic acids.

In some embodiments, the method includes releasing the probe from the connected probe or ligation product. In some embodiments, the releasing includes the use of heat. The probe can be designed to be released (e.g., melted) from the ligation product based on the probe length, temperature and ionic strength. This approach utilizes the ligation of the splint oligonucleotide to increase the length when there is a specific probe and miRNA interaction. Thus, ligation occurs when there is a specific interaction and due to the shorter length of the miRNA molecules even single base mismatches can impact the likelihood of a successful ligation, imparting the specificity to this readout. Temperature, splint oligonucleotide length, and ionic strength can be tuned to ensure specific ligation and selective melting or retention of different species (see, e.g., Kawasaki, M. and Masayuki, O., Effects of chain length, temperature, and ionic strength on association and dissociation thermodynamics of DNA, Chemical Thermodynamics and Thermal Analysis, Vols. 3-4 (2021)).

In some embodiments, the method includes extending the capture probe using the connected probe or ligation product as a template, thereby generating an extended capture probe. Thus, the extended capture probe can include a sequence complementary to the connected probe or ligation product. In some embodiments, the extending includes the use of a polymerase. In some embodiments, the method includes extending the connected probe or ligation product, thereby generating an extended connected probe or extended ligation product, where the extended connected probe or extended ligation product includes a sequence complementary to the capture probe.

In some embodiments, the nucleic acid capture sequence of the splint oligonucleotide includes a homopolymeric sequence or a fixed sequence. In some embodiments, the homopolymeric sequence is a poly(A) sequence.

In some embodiments, the non-coding RNA is a siRNA. In some embodiments, the non-coding RNA is a miRNA. In some embodiments, the miRNA is a pri-miRNA. In some embodiments, the miRNA is a pre-miRNA.

In some embodiments, the method includes, use of one or more Argonaute proteins. The Argonaute proteins may be complexed with the non-coding RNA. Argonaute proteins are the active part of the RNA-induced silencing complex (RISC). The RISC complex is responsible for gene silencing (e.g., RNA interference). Argonaute proteins are known to bind different classes of non-coding RNAs, including miRNA, siRNA and piwi-interacting RNAs (piRNA). Argonaute proteins include a small RNA that hybridizes to mRNA in the biological sample. Upon hybridization to the mRNA, mRNA cleavage, translation inhibition, and/or initiation of mRNA decay occurs. There are several Argonaute proteins found across plants and animals, including humans. More specifically AGO1, AGO2, AGO3, and AGO4 are capable of loading non-coding RNAs, however, AGO2 possesses the endonuclease activity and thus the RNA interference dependent gene silencing in humans. In some embodiments, the one or more Argonaute proteins includes one or more of: AGO1, AGO2, AGO3, and AGO4. In some embodiments, the one or more Argonaute proteins lack cleavage activity (e.g., catalytically dead Argonaute). For example, in some embodiments, the Argonaute does not cleave the probe and/or splint oligonucleotide hybridized to the non-coding RNA (e.g., miRNA).

In some embodiments, the probes, probe pairs, splint oligonucleotides, and other nucleic acids used to capture or hybridize to non-coding RNA include one or more locked nucleic acids. Locked nucleic acid, or LNAs are modified RNA monomers which include a methylene bridge bond linking the 2′ oxygen to the 4′ carbon of the RNA pentose ring. The bridge bond fixes the pentose ring in 3′-endo conformation. In some embodiments, the capture domain (e.g., a capture domain of a first capture probe, a capture domain of a second capture probe, etc.) includes 1, 2, 3, 4, 5, 6, 7, 8, 9, 10, 11, 12, 13, 14, 15, or more LNAs. In some embodiments, the probe contacted with the biological sample to detect ncRNA includes one or more locked nucleic acids in a particular region, e.g., to help improve hybridization specificity. For example, in some embodiments, a portion of the probe including a nucleic acid sequence capable of hybridizing to at least a portion of a non-coding RNA includes one or more locked nucleic acids.

In some embodiments, e.g., before or during step (c), the method includes using one or more minor groove binders. Minor groove binders are crescent shaped molecules that selectively bind non-covalently to the minor groove of DNA e.g., a shallow furrow in the DNA helix. Minor groove binders can be neutral e.g., bind non-selectively or sequence specific. Minor groove binders can also stabilize double-stranded DNA. In some embodiments, the one or more minor groove binders includes one or more of duocarmycin A, Chromomycin A3, and alkamin.

In some embodiments, the method includes imaging the biological sample. In some embodiments, the method includes staining the biological sample. In some embodiments, the staining includes hematoxylin and/or eosin staining. In some embodiments, the staining includes the use of a detectable label selected from the group consisting of a radioisotope, a fluorophore, a chemiluminescent compound, a bioluminescent compound, or a combination thereof.

After placement of the biological sample onto the substrate, biological samples can be stained using a wide variety of stains and staining techniques. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample.

The sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample. In some embodiments, immunofluorescence or immunohistochemistry protocols (direct and indirect staining techniques) can be performed as a part of, or in addition to, the exemplary spatial workflows presented herein.

In some embodiments, the permeabilizing includes the use of an organic solvent, a detergent, and an enzyme, or a combination thereof. In some embodiments, permeabilizing includes the use of an endopeptidase, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof. In some embodiments, permeabilizing includes and endopeptidase, such as pepsin, or proteinase K.

In some embodiments, the capture probe includes a cleavage domain, one or more functional domains, a unique molecular identifier, or combinations thereof. In some embodiments, the capture domain of the capture probe includes a homopolymeric sequence. In some embodiments, the homopolymeric sequence is a poly(T) sequence. In some embodiments, the capture domain of the capture probe includes a fixed sequence.

In some embodiments, the array includes one or more features selected from the group consisting of: a spot, an inkjet spot, a masked spot, a pit, a post, a well, a ridge, a divot, a hydrogel pad, and a bead.

In some embodiments, the method includes migrating the probe to the array e.g., before step (d). In some embodiments, the method includes migrating the extended probe to the array. In some embodiments, the method includes migrating the ligation product to the array. In some embodiments, the method includes migrating the capture probe to the biological sample e.g., for hybridization to the connected probe or ligation product. For example, the capture probe can be cleaved at a cleavage domain within the capture probe to release the capture probe from the array. In some embodiments, the migrating includes electrophoresis.

In some embodiments, the biological sample is a tissue sample. In some embodiments, the tissue sample is a fresh-frozen tissue sample. In some embodiments, the tissue sample is a fixed tissue sample, and optionally, the fixed tissue sample is a formalin-fixed paraffin-embedded tissue sample, an acetone-fixed tissue sample, a methanol-fixed tissue sample, or a paraformaldehyde-fixed tissue sample. In some embodiments, the biological sample is a tissue section. In some embodiments, the tissue section is a fresh-frozen tissue section. In some embodiments, the tissue section is a fixed tissue section. In some embodiments, the fixed tissue section is a formalin-fixed paraffin-embedded tissue section, an acetone-fixed tissue section, a methanol-fixed tissue section, or a paraformaldehyde-fixed tissue section.

(iii) Exemplary Non-Coding RNAs

Since detection of the non-coding RNAs (ncRNA) relies on the principle of nucleic acid complementarity and hybridization, any suitable ncRNA can be detected in accordance with the disclosed methods. For example, the probes can be designed such that they are complementary to a target ncRNA. In some embodiments, the extent of complementarity between the probe and target ncRNA can be at least 70%, at least 75%, at least 80%, at least 85%, at least 90, at least 95%, at least 96%, at least 97%, at least 98%, at least 99%, or 100%.

Many miRNAs are grouped into families based upon high sequence homology. In particular, nucleotide positions 2 through 7 from the 5′ end (termed the “seed”) are generally 100% homologous. See, Lewis, B P. et al., Prediction of Mammalian MicroRNA Targets. Cell. 115:787-798 (2003). The remaining nucleotides may differ by as few as a single nucleotide. This nucleotide difference may occur at any other position in the miRNA. Thus, in some embodiments, the probes can be designed to detect an individual miRNA or a miRNA family (e.g., where the ncRNA-binding domain is complementary to the seed sequence of a miRNA family, but cannot distinguish between several members of the miRNA family).

In some embodiments, the disclosed methods allow for detection of a ncRNA (e.g., miRNA) associated with one or more diseases. For example, studies have shown that differential miRNA expression occurs in cancerous and non-cancerous tissues. miRNAs represent 1% of the mammalian genome but more than 50% of miRNA genes are located within regions associated with amplification, deletion and translocation in cancer.

Suitable miRNAs that can be detected in accordance with the methods disclosed herein include, without limitation, MIR21, MIR22, MIR23C, MIR31, MIR32, MIR96, MIR122, MIR126, MIR127, MIR134, MIR136, MIR137, MIR139, MIR140, MIR142, MIR143, MIR144, MIR145, MIR149, MIR150, MIR155, MIR182, MIR183, MIR184, MIR185, MIR186, MIR187, MIR191, MIR197, MIR198, MIR202, MIR205, MIR210, MIR214, MIR217, MIR221, MIR222, MIR223, MIR224, MIR296, MIR297, MIR298, MIR299, MIR300, MIR302E, MIR324, MIR325, MIR326, IR328, MIR330, MIR331, MIR335, MIR337, MIR338, MIR339, MIR340, MIR342, MIR345, MIR346, MIR361, MIR367, MIR370, MIR375, MIR378C, MIR378F, MIR378G, MIR378H, MIR378I, MIR378J, MIR383, MIR384, MIR412, MIR422A, MIR423, MIR425, MIR 431, MIR 432, MIR 433, MIR 448, MIR452, MIR455, MIR466, MIR483, MIR 484, MIR485, MIR488, MIR489, MIR490, MIR491, MIR492, MIR493, MIR498, MIR499A, MIR499B, MIR504, MIR505, MIR541, MIR542, MIR548BC, MIR5480, MIR549A, MIR552, MIR553, MIR554, MIR555, MIR556, MIR557, MIR558, MIR559, MIR561, MIR562, MIR563, MIR564, MIR567, MIR568, MIR569, MIR571, MIR572, MIR573, MIR574, MIR575, MIR576, MIR577, MIR578, MIR580, MIR581, MIR582, MIR583, MIR584, MIR585, MIR586, MIR587, MIR588, MIR589, MIR590, MIR591, MIR592, MIR593, MIR595, MIR596, MIR597, MIR598, MIR599, MIR600, MIR601, MIR602, MIR604, MIR605, MIR606, MIR607, MIR608, MIR609, MIR610, MIR611, MIR612, MIR613, MIR614, MIR615, MIR616, MIR617, MIR618, MIR619, MIR620, MIR621, MIR622, MIR623, MIR624, MIR625, MIR626, and LET-7 (including family members Let-7a, b, c, d, e, f, g, i, MIR-98 and MIR-202).

### (iv) Library Preparation

In some embodiments, the products derived from non-coding RNA, or complements thereof, and/or amplicons of such products (e.g., extended capture probes, extended connected probes, extended ligation products), can be prepared for downstream applications, such as generation of a sequencing library and next-generation sequencing. Generating sequencing libraries are known in the art. For example, the products derived from non-coding RNA can be purified and collected for downstream amplification steps. The amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the non-coding RNA sequence.

Alternatively or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flow cell (e.g., on Illumina sequencing instruments). Additionally, i7 and i5 can index sequences be added as sample indexes if multiple libraries are to be pooled and sequenced together. Further, Read 1 and Read 2 sequences can be added to the library preparation for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

### (v) Sandwich Processes

In some embodiments, one or more non-coding RNAs from the biological sample are released from the biological sample and captured by any of the methods described herein. The resulting products (e.g., ligation product, connected probes, probes, extended probes, etc.) can be captured by capture probes immobilized on a substrate. In some embodiments, the release and migration of the aforementioned products to the substrate comprising the array of capture probes occurs in a manner that preserves the original spatial context of the non-coding RNAs in the biological sample. It is also contemplated that capture probes can be cleaved (e.g., via a cleavage domain such as a USER enzyme site or UV cleavable domain) to be released from the array and migrate to the biological sample. In some embodiments, the release and migration of capture probes to the biological sample occurs in a manner that preserves the original spatial context of the capture probe in the array. In some embodiments, the biological sample is mounted on a first substrate and the substrate comprising the array of capture probes is mounted on a second substrate. In some embodiments, the alignment of the first substrate and the second substrate is facilitated by a sandwiching process. Accordingly, described herein are methods, compositions, and kits for sandwiching together the first substrate as described herein with a second substrate having an array with capture probes.

In some embodiments, the sandwiching process may be facilitated by a device, sample holder, sample handling apparatus, or system described in, e.g., US. Patent Application Pub. No. 20210189475, PCT/US2021/036788, or PCT/US2021/050931.

In some embodiments, the first and second substrates are placed in a substrate holder (e.g., an array alignment device) configured to align the biological sample and the array. In some embodiments, the device comprises a sample holder. In some embodiments, the sample holder includes a first member and a second member that receive a first substrate and a second substrate, respectively. The device can include an alignment mechanism that is connected to at least one of the members and aligns the first and second members. Thus, the devices of the disclosure can advantageously align the first substrate and the second substrate and any samples, barcoded probes, or permeabilization reagents that may be on the surface of the first and second substrates.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the non-coding RNA from the biological sample.

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The alignment mechanism may be configured to move the second member along the axis orthogonal to the second plane and/or move the first member along an axis orthogonal to the first plane.

In some embodiments, the alignment mechanism includes a linear actuator. In some embodiments, the alignment mechanism includes one or more of a moving plate, a bushing, a shoulder screw, a motor bracket, and a linear actuator. The moving plate may be coupled to the first member or the second member. The alignment mechanism may, in some cases, include a first moving plate coupled to the first member and a second moving plate coupled to the second member. In some embodiments, the linear actuator is configured to move the second member along an axis orthogonal to the plane of the first member and/or the second member. For example, the moving plate may be coupled to the second member and adjust the separation distance along a z axis (e.g., orthogonal to the second substrate) by moving the moving plate up in a superior direction toward the first substrate. In some embodiments, the linear actuator is configured to move the first member along an axis orthogonal to the plane of the first member and/or the second member. The movement of the moving plate may be accomplished by the linear actuator configured to move the first member and/or the second member at a velocity. The velocity may be controlled by a controller communicatively coupled to the linear actuator. In some embodiments, the linear actuator is configured to move the first member, the second member, or both the first member and the second member at a velocity of at least 0.1 mm/sec (e.g., at least 0.1 mm/sec to 2 mm/sec). In some aspects, the velocity may be selected to reduce or minimize bubble generation or trapping within the reagent medium. In some embodiments, the linear actuator is configured to move the first member, the second member, or both the first member and the second member with an amount of force of at least 0.1 lbs (e.g., between 0.1-4.0 pounds of force).

In some aspects, the velocity of the moving plate (e.g., closing the sandwich) may affect bubble generation or trapping within the reagent medium. It may be advantageous to minimize bubble generation or trapping within the reagent medium during the “sandwiching” process, as bubbles can interfere with the migration of products derived from non-coding RNA through the reagent medium to the array. In some embodiments, the closing speed is selected to minimize bubble generation or trapping within the reagent medium. In some embodiments, the closing speed is selected to reduce the time it takes the flow front of the reagent medium from an initial point of contact with the first and second substrate to sweep across the sandwich area (also referred to herein as “closing time”). In some embodiments, the closing speed is selected to reduce the closing time to less than about 1100 milliseconds (ms). In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less.

Further details on support devices, sample holders, sample handling apparatuses, or systems for implementing a sandwiching process are described in, e.g., PCT Publ. No. WO 2021/0189475 and PCT/US2021/050931, each of which are incorporated by reference in their entireties.

Non-coding RNAs within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein)

Provided herein are methods for delivering a fluid to a biological sample disposed on an area of a first substrate and an array disposed on a second substrate.

In some embodiments, the sandwich configuration described herein between a first substrate comprising a biological sample and a second substrate comprising a spatially barcoded array may include a reagent medium comprising NaCl, ethylene carbonate, and/or glycerol to fill a gap. It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates during a permeabilization step.

In some aspects, it may be possible to reduce or eliminate bubble formation between the slides using a variety of filling methods and/or closing methods.

In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

Robust fluidics in the sandwich making described herein may preserve spatial information by reducing or preventing deflection of molecules as they move from the tissue slide to the capture slide.

Further details on angled closure workflows, and devices and systems for implementing an angled closure workflow, are described in PCT/US2021/036788 and PCT/US2021/050931, which are hereby incorporated by reference in their entireties. Additional configurations for reducing or eliminating bubble formation, and/or for reducing unwanted fluid flow, are described in PCT/US2021/036788, which is hereby incorporated by reference in its entirety. Suitable permeabilization agents include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). Exemplary permeabilization reagents are described in in PCT Patent Application Publication No. WO 2020/123320, which is incorporated by reference in its entirety.

Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Exemplary lysis reagents are described in PCT Patent Application Publication No. WO 2020/123320, which is incorporated by reference in its entirety.

Exemplary proteases include, e.g., pepsin, trypsin, pepsin, elastase, and proteinase K. Exemplary proteases are described in PCT Patent Application Publication No. WO 2020/123320, which is incorporated by reference in its entirety. Exemplary detergents include sodium dodecyl sulfate (SDS), sarkosyl, saponin, Triton X-100™, and Tween-20™. Exemplary detergents are described in PCT Patent Application Publication No. WO 2020/123320, which is incorporated by reference in its entirety.

In some embodiments, the reagent medium comprising NaCl, ethylene carbonate, and/or glycerol also comprises a nuclease. In some embodiments, the nuclease comprises am RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNase, and a sodium salt thereof.

The sample holder is compatible with a variety of different schemes for contacting the aligned portions of the biological sample and array with the reagent medium to promote capture of the products derived from non-coding RNA. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include, but are not limited to, spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some embodiments, following initial contact between sample and a permeabilization agent, the permeabilization agent can be removed from contact with sample (e.g., by opening sample holder) before complete permeabilization of sample. For example, in some embodiments, only a portion of sample is permeabilized, and only a portion of the products derived from non-coding RNA in sample may be captured by feature array. In some instances, the reduced amount of products derived from non-coding RNA captured and available for detection can be offset by the reduction in lateral diffusion that results from incomplete permeabilization of sample. In general, the spatial resolution of the assay is determined by the extent of products derived from non-coding RNA diffusion in the transverse direction (i.e., orthogonal to the normal direction to the surface of sample). The larger the distance between the sample on the first substrate and the feature array on the second substrate, the greater the extent of diffusion in the transverse direction, and the concomitant loss of resolution. Non-coding RNAs liberated from a portion of the sample closest to the feature array have a shorter diffusion path, and therefore do not diffuse as far laterally as products derived from non-coding RNA from portions of the sample farthest from the feature array. As a result, in some instances, incomplete permeabilization of the sample (by reducing the contact interval between the permeabilization agent and the sample) can be used to maintain adequate spatial resolution in the assay.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about-10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, non-coding RNAs that are released from the permeabilized tissue of the sample diffuse to the surface of the second substrate and are captured on the array (e.g., barcoded probes) of the second substrate. In a fourth step, the first substrate and the second substrate are separated (e.g., pulled apart) and temperature control is stopped.

In some embodiments, where either the first substrate or substrate second (or both) includes wells, a permeabilization solution can be introduced into some or all of the wells, and then the sample and the feature array can be contacted by closing the sample holder to permeabilize the sample. In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of products derived from non-coding RNA from the sample to the array.

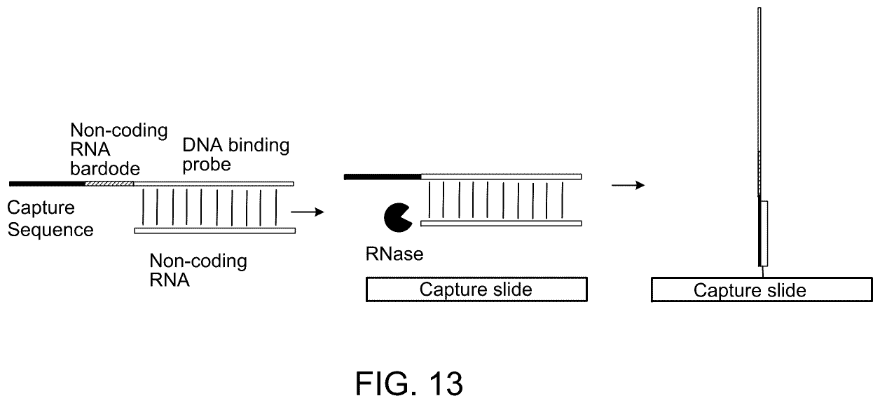

In certain embodiments, different permeabilization agents or different concentrations of permeabilization agents can be infused into array features (e.g., beads) or into a hydrogel layer as described above. By locally varying the nature of the permeabilization reagent(s), the process of products derived from non-coding RNA capture from the sample can be spatially adjusted. In some instances, migration of the products derived from non-coding RNA from the biological sample to the second substrate is passive (e.g., via diffusion). Alternatively, in certain embodiments, migration of the products derived from non-coding RNA from the biological sample is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher capture efficiency of products derived from non-coding RNA and better spatial fidelity of captured products derived from non-coding RNA (e.g., on a feature array) than random diffusion onto matched substrates without the application of an electric field (e.g., via manual alignment of the two substrates). Exemplary methods of electrophoretic migration are described in WO 2020/176788, including at FIGS. 13-15, 24A-24B, and 25A-25C, which is hereby incorporated by reference in its entirety.

Loss of spatial resolution can occur when products derived from non-coding RNA migrate from the sample to the feature array and a component of diffusive migration occurs in the transverse (e.g., lateral) direction, approximately parallel to the surface of the first substrate on which the sample is mounted. To address this loss of resolution, in some embodiments, a permeabilization agent deposited on or infused into a material with anisotropic diffusion can be applied to the sample or to the feature array. The first and second substrates are aligned by the sample holder and brought into contact. A permeabilization layer that includes a permeabilization solution infused into an anisotropic material is positioned on the second substrate.

In some embodiments, the feature array can be constructed atop a hydrogel layer infused with a permeabilization agent. The hydrogel layer can be mounted on the second substrate, or alternatively, the hydrogel layer itself may function as the second substrate. When the first and second substrates are aligned, the permeabilization agent diffuses out of the hydrogel layer and through or around the feature array to reach the sample. Products derived from non-coding RNA from the sample migrate to the feature array. Direct contact between the feature array and the sample helps to reduce lateral diffusion of the products derived from non-coding RNA, mitigating spatial resolution loss that would occur if the diffusive path of the products derived from non-coding RNAs was longer.

Spatial analysis workflows can include a sandwiching process described herein. In some embodiments, the workflow includes provision of the first substrate comprising the biological sample. In some embodiments, the workflow includes, mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, image are captured using high resolution techniques (e.g., having 300 dots per square inch (dpi) or greater). For example, images can be captured using brightfield imaging (e.g., in the setting of hematoxylin or H&E stain) or using fluorescence microscopy to detect adhered labels. In some instances, high resolution images are captured temporally using e.g., confocal microscopy. In some instances, a low resolution image is captured. A low resolution image (e.g., images that are about 72 dpi and normally have an RGB color setting) can be captured at any point of the workflow, including but not limited to staining, destaining, permeabilization, sandwich assembly, and migration of the products derived from non-coding RNA. In some instances, a low resolution image is taken during permeabilization of the biological sample.

In some instances, the biological samples can be destained. In some instances, destaining occurs prior to permeabilization of the biological sample. By way of example only, H&E staining can be destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

In some instances, after the sandwiching process the first substrate and the second substrate are separated (e.g., such that they are no longer aligned in a sandwich configuration, also referred to herein as opening the sandwich). In some embodiments, subsequent analysis (e.g., cDNA synthesis, library preparation, and sequences) can be performed on the captured products derived from non-coding RNA after the first substrate and the second substrate are separated.

In some embodiments, the process of transferring the products derived from non-coding RNA from the first substrate to the second substrate is referred to interchangeably herein as a “sandwich process,” “sandwiching process,” or “sandwiching”. The sandwich process is further described in PCT Patent Application Publication No. WO 2020/123320, PCT/US2021/036788, and PCT/US2021/050931, which are incorporated by reference in their entireties.

### (vi) Compositions and Kits

Also disclosed herein are kits and compositions used for any one of the methods disclosed herein. In some instances, the kits or compositions are used for determining the location of a non-coding RNA in a biological sample. Thus, provided herein are kits including: (a) an array including a plurality of capture probes, where a capture probe of the plurality of capture probes includes: (i) a spatial barcode and (ii) a capture domain; and (b) a plurality of probes, where a probe of the plurality of probes includes (i) a nucleic acid sequence complementary to a non-coding RNA and (ii) a nucleic acid capture sequence capable of hybridizing to at least a portion of the capture domain.

Also provided herein are kits including: (a) an array including a plurality of capture probes, where a capture probe of the plurality of capture probes includes: (i) a spatial barcode and (ii) a capture domain; (b) a plurality of probes, where a probe of the plurality of probes includes a nucleic acid sequence capable of hybridizing to at least a portion of a non-coding RNA; (c) a reverse transcriptase; and (d) a plurality of adaptors, where an adaptor of the plurality of adaptors includes: (i) a nucleic acid sequence capable of hybridizing to a polynucleotide sequence including at least three nucleotides added to an extended version of the probe and (ii) a nucleic acid sequence complementary to a nucleic acid capture sequence, where the nucleic acid capture sequence is capable of hybridizing to at least a portion of the capture domain of the capture probe.

Also provided herein are kits including: (a) an array including a plurality of capture probes, where a capture probe of the plurality of capture probes includes: (i) a spatial barcode and (ii) a capture domain; and (b) a plurality of first probes and a plurality of second probes, where a first probe of the plurality of first probes and a second probe of the plurality of second probes include sequences that are capable of hybridizing to a non-coding RNA, and where the second probe includes a nucleic acid capture sequence that is capable of hybridizing to at least a portion of the capture domain of the capture probe.

Also provided herein are kits including: (a) an array including a plurality of capture probes, where a capture probe of the plurality of capture probes includes: (i) a spatial barcode and (ii) a capture domain; (b) a plurality of probes, where a probe of the plurality of probes includes: (i) a nucleic acid sequence capable of hybridizing to at least a portion of a non-coding RNA and (ii) a ligation handle; and (c) a plurality of splint oligonucleotides, where a splint oligonucleotide of the plurality of splint oligonucleotides includes: (i) a sequence capable of hybridizing to at least a portion of the ligation handle and (ii) a nucleic acid capture sequence capable of hybridizing to at least a portion of the capture domain of the capture probe.

The kits described herein can further include one or more enzymes. For example, the kit can include a reverse transcriptase, a polymerase such as a DNA polymerase, a ligase, and combinations thereof.

In some embodiments, the kit includes a plurality of dNTPs. In some embodiments, the plurality of dNTPs includes a plurality of dCTPs, however, any dNTP can be included such as dATP, dGTP, and/or dTTP.

In some embodiments, the kit includes one or more minor groove binders. In some embodiments, the one or more minor grove binders includes one or more of duocarmycin A, Chromomycin A3, and alkamin.

In some embodiments, the kit includes one or more permeabilization reagents. In some embodiments, the one or more permeabilization reagents includes one or more of a protease, a lipase, an RNase, a detergent, and a surfactant.

In some embodiments, the kit includes one or more Argonaute proteins. In some embodiments, the one or more Argonaute proteins includes one or more of: AGO1, AGO2, AGO3, and AGO4.

Also provided herein are compositions including: (a) an array including a plurality of capture probes, where a capture probe of the plurality of capture probes includes: (i) a spatial barcode and (ii) a capture domain; and (b) a probe hybridized to a non-coding RNA, where the probe includes: (i) a nucleic acid sequence hybridized to at least a portion of the non-coding RNA and (ii) a nucleic acid capture sequence capable of hybridizing to at least a portion of the capture domain of the capture probe.

In some embodiments, the nucleic acid capture sequence is hybridized to at least a portion of the capture domain of the capture probe. In some embodiments, the capture probe has been extended by using the probe as a template. In some embodiments, the probe has been extended by using the capture probe as a template.

Also provided herein are compositions including: (a) an array including a plurality of capture probes, where a capture probe of the plurality of capture probes includes: (i) a spatial barcode and (ii) a capture domain; (b) a plurality of probes, where a probe of the plurality of probes includes a nucleic acid sequence capable of hybridizing to at least a portion of a non-coding RNA; (c) a reverse transcriptase; and (d) a plurality of adaptors, where an adaptor of the plurality of adaptors includes: (i) a nucleic acid sequence capable of hybridizing to a polynucleotide sequence including at least three nucleotides added to an extended version of the probe and (ii) a nucleic acid sequence complementary to a nucleic acid capture sequence, where the nucleic acid capture sequence is capable of hybridizing to at least a portion of the capture domain.

In some embodiments, the probe has been extended using the non-coding RNA as an extension template to generate the extended version of the probe. In some embodiments, the extended probe includes the polynucleotide sequence at its 3′ end. In some embodiments, the nucleic acid sequence (i) of the adaptor is hybridized to the polynucleotide sequence. In some embodiments, the extended probe has been extended using the adaptor as an extension template, thereby incorporating the nucleic acid capture sequence into the extended probe. In some embodiments, the nucleic acid capture sequence of the extended probe is hybridized to at least a portion of the capture domain of the capture probe.

In some embodiments, the capture probe has been extended using the extended probe as an extension template. In some embodiments, the extended probe has been extended using the capture probe as an extension template.

Also provided herein are compositions including: (a) an array including a plurality of capture probes, where a capture probe of the plurality of capture probes includes: (i) a spatial barcode and (ii) a capture domain; (b) a plurality of first probes, where a first probe of the plurality of first probes includes a sequence that is capable of hybridizing to at least a portion of a non-coding RNA; (c) a plurality of second probes, where a second probe of the plurality of second probes includes: (i) a sequence that is capable of hybridizing to at least a portion of the non-coding RNA and (ii) a nucleic acid capture sequence that is capable of hybridizing to at least a portion of the capture domain of the capture probe; and (d) a ligase.

In some embodiments, the first probe and the second probe have been ligated to generate a ligation product. In some embodiments, the nucleic acid capture sequence of the ligation product is hybridized to at least a portion of the capture domain of the capture probe. In some embodiments, the capture probe has been extended using the ligation product as a template. In some embodiments, the ligation product has been extended using the capture probe as a template.

Also provided herein are compositions including: (a) an array including a plurality of capture probes, where a capture probe of the plurality of capture probes includes: (i) a spatial barcode and (ii) a capture domain; (b) a plurality of probes, where a probe of the plurality of probes includes: (i) a nucleic acid sequence capable of hybridizing to at least a portion of a non-coding RNA and (ii) a ligation handle; and (c) a plurality of splint oligonucleotides, where a splint oligonucleotide of the plurality of splint oligonucleotides includes: (i) a sequence capable of hybridizing to at least a portion of the ligation handle and (ii) a nucleic acid capture sequence capable of hybridizing to at least a portion of the capture domain of the capture probe.

In some embodiments, the non-coding RNA and the splint oligonucleotide have been ligated, thereby generating a ligation product. In some embodiments, the nucleic acid capture sequence of the ligation product is hybridized to the capture domain of the capture probe. In some embodiments, the capture probe has been extended using the ligation product as an extension template.

In some embodiments, the ligation product has been extended using the capture probe as an extension template. In some embodiments, the non-coding RNA is a siRNA. In some embodiments, the non-coding RNA is a miRNA. In some embodiments, the miRNA is a pri-miRNA. In some embodiments, the miRNA is a pre-miRNA.

In some embodiments, the capture sequence includes a homopolymeric sequence. In some embodiments, the homopolymeric sequence includes a poly(A) sequence. In some embodiments, the nucleic acid capture sequence includes a fixed sequence.

In some embodiments, the capture domain includes a poly(T) sequence. In some embodiments, the capture domain includes a fixed sequence.

In some embodiments, the array includes one or more features selected from the group consisting of: a spot, an inkjet spot, a masked spot, a pit, a post, a well, a ridge, a divot, a hydrogel pad, and a bead.

In some embodiments, the composition includes a reverse transcriptase. In some embodiments, the composition includes a polymerase. In some embodiments, the polymerase is a DNA polymerase. In some embodiments, the composition includes a ligase.

In some embodiments, the probe includes one or more locked nucleic acids (e.g., any of the locked nucleic acids described herein). In some embodiments, the composition includes one or more minor grove binders. In some embodiments, the one or more minor groove binders includes one or more of duocarmycin A, Chromomycin A3, and alkamin.

In some embodiments, the composition includes one or more Argonaute proteins. In some embodiments, the one or more Argonaute proteins includes one or more of AGO1, AGO2, AGO3, and AGO4.

In some embodiments, the composition includes one or more permeabilization reagents including one or more of a protease, a lipase, an RNase, a detergent, and a surfactant.

In some embodiments, the composition includes a biological sample. In some embodiments, the biological sample is a tissue section. In some embodiments, the tissue section is a fresh-frozen tissue section. In some embodiments, the tissue section is a fixed tissue section. In some embodiments, the fixed tissue section is a formalin-fixed paraffin-embedded tissue section, an acetone-fixed tissue section, a methanol-fixed tissue section, or a paraformaldehyde-fixed tissue section. In some embodiments, the biological sample is a tissue sample. In some embodiments, the tissue sample is a fixed tissue sample.

### EXAMPLES

### Example 1. Spatial Capture of Non-Coding RNA

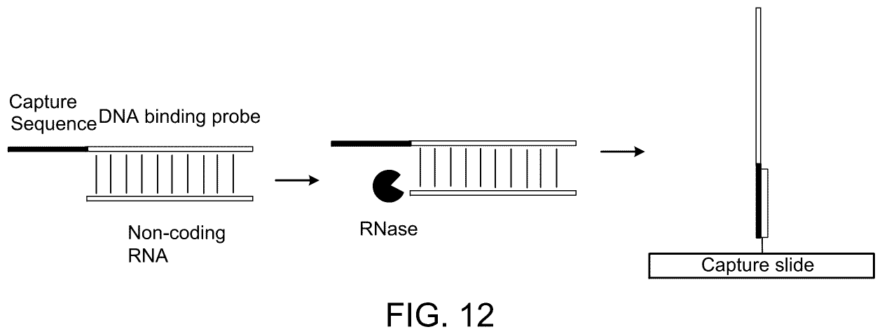

FIG. 12 is a schematic diagram showing an exemplary workflow for spatial capture of non-coding RNA. From left to right the FIG. 12 shows a nucleic acid probe hybridized to a non-coding RNA (e.g., any of the non-coding RNAs described herein). The probe also includes a nucleic acid capture sequence that is substantially complementary to a capture domain of a capture probe (e.g., any of the capture probes described herein) immobilized on a substrate (i.e., “capture slide”). Next, the biological sample is contacted with an endonuclease such as an RNase (e.g., any of the RNases described herein). The RNase degrades the non-coding RNA leaving the probe to bind (e.g., hybridize) to the capture domain of the capture probe (far right). Once the probe hybridizes to the capture domain, the probe can be extended using the capture probe as a template and/or the capture probe can be extended using the probe as a template (i.e., an extended capture probe). The extension reaction(s) incorporates the spatial barcode, or a complement thereof, into the extended product(s). The extended products can be optionally further amplified and processed (e.g., sequenced) to determine the location of the non-coding RNA in the biological sample.

In some examples, the probe includes one or more locked nucleic acids (e.g., in the non-coding RNA-binding domain) as described herein to increase probe specificity for non-coding RNAs. In some examples, a minor groove binder (e.g., any of the exemplary minor groove binders described herein) is added before, simultaneously, or after the probe is contacted with the biological sample. In some examples, the probe includes one or more phosphorothioate bonds to protect the probe from degradation.

FIG. 12 shows an embodiment where capture from the biological sample occurs on the capture slide (e.g., the biological sample is placed on the capture slide), however, it is appreciated that the biological sample can be on a different substrate, the probe, and optionally a minor groove binder are contacted with the biological sample, followed by bringing the substrate with the biological sample in a sandwich configuration as described herein with the spatial array including the plurality of capture probes.

FIG. 13 is a schematic diagram showing an exemplary workflow for spatial capture of non-coding RNA similar to the workflow described in FIG. 12 above. In the embodiment shown in FIG. 13 the probe includes an additional nucleic acid sequence identified as a non-coding RNA barcode. The non-coding barcode is a unique sequence that corresponds to the non-coding RNA to which the probe is designed to hybridize. The non-coding RNA barcode can function as an additional verification of the non-coding RNA identified if the probe is degraded through nuclease activity (i.e., the portion of the probe complementary to the non-coding RNA).

### Example 2. Spatial Capture of Non-Coding RNA Via In Situ Reverse Transcription

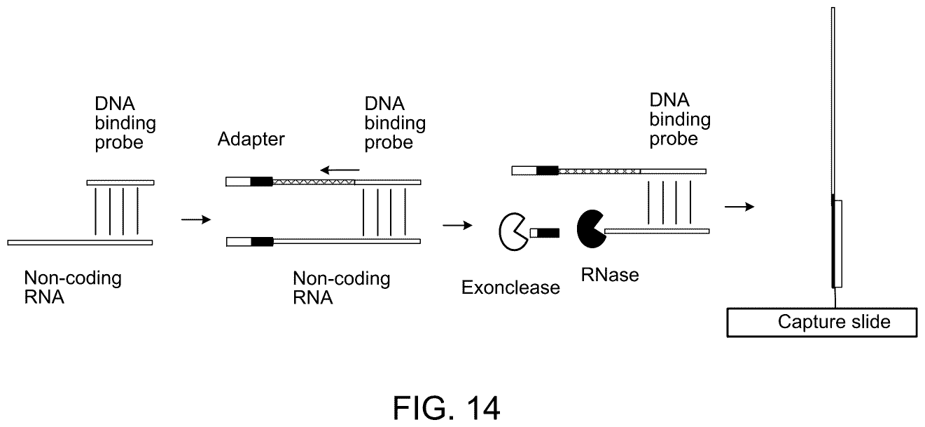

FIG. 14 is a schematic diagram showing an exemplary workflow for spatial capture of non-coding RNA via in situ reverse transcription. From left to right FIG. 14 shows a nucleic acid binding probe hybridized to at least a portion of the targeted non-coding RNA. Next, the probe is extended (e.g., reverse transcribed) using the non-coding RNA as a template and non-templated nucleotides are added to the extended probe. An adaptor (e.g., a template switch oligonucleotide) including a sequence complementary to the added non-templated nucleotides hybridizes to the non-templated nucleotides and the extended probe is further extended. The adaptor includes a sequence complementary to a capture sequence, such that when the extended probe is further extended a capture sequence is generated. The generated capture sequence is substantially complementary to a capture domain of a capture probe immobilized on a substrate (i.e., “capture slide”).

After incorporation of the capture sequence into the extended probe, the non-coding RNA and the adaptor can be removed from the extended probe including the capture sequence. Removal includes the use of an endonuclease such as an RNase (e.g., any of the RNases described herein) and T7 exonuclease. The RNase can digest away the non-coding RNA and the T7 exonuclease can digest away the adaptor. T7 exonuclease hydrolyzes duplexed DNA in 5′ to 3′ direction and is used to generate single-stranded DNA templates. In some examples, 5′ end of the adaptor is phosphorylated. In such examples, lambda exonuclease can be used instead of T7 exonuclease. After removal of the adaptor and the non-coding RNA, the single-stranded extended probe including the capture sequence a complementary sequence of the non-coding RNA hybridizes to the capture domain of the capture probe on the substrate. The extended probe and/or the capture probe can be extended thereby incorporating the spatial barcode of the capture probe, or a complement thereof, into the extended probe and/or the extended capture probe. Optionally, the extended capture probe or the extended probe can be amplified and further processed (e.g., sequenced) to determine the location of the non-coding RNA in the biological sample.

In some examples, the probe includes one or more locked nucleic acids as described herein to increase probe specificity for non-coding RNAs. In some examples, a minor groove binder (e.g., any of the exemplary minor groove binders described herein) is added before, simultaneously, or after the probe is contacted with the biological sample. In some examples, the probe includes one or more phosphorothioate bonds to protect the probe from degradation.

FIG. 14 shows an embodiment where capture from the biological sample occurs on the capture slide (e.g., the biological sample is placed on the capture slide), however, it is appreciated that the biological sample can be on a different substrate, the probe, and optionally a minor groove binder are contacted with the biological sample, followed by bringing the substrate with the biological sample in a sandwich configuration as described herein with the array including the plurality of capture probes.

### Example 3. Spatial Capture of Non-Coding RNA Via Templated Ligation

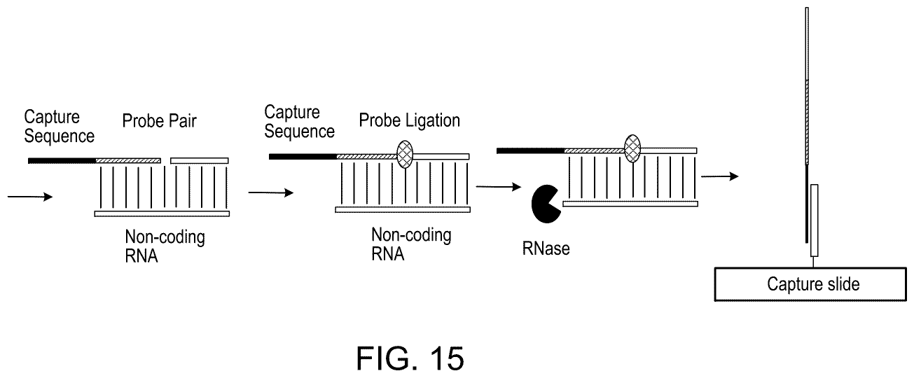

FIG. 15 is a schematic diagram showing an exemplary workflow for spatial capture of non-coding RNA via a templated ligation reaction. From left to right FIG. 15 shows a probe pair hybridized to a non-coding RNA in a biological sample. In some examples, the probes in the probe pair hybridize directly adjacent to one another. In some examples, the probes in the probe pair hybridize to non-adjacent sequences and a gap-filling reaction is performed with a polymerase to extend the first probe or the second probe. In some examples, one of the probes of the probe pair includes a capture sequence that is substantially complementary to a capture domain of a capture probe immobilized on a substrate. After hybridization to the non-coding RNA the probes are ligated together via the use of a ligase (e.g., any of the ligases described herein) thereby generating a ligation product. Next, the biological sample is contacted with an endonuclease such as an RNase (e.g., any of the RNases described herein) to degrade the non-coding RNA. Once the non-coding RNA is degraded, the single-stranded ligation product hybridizes to the capture probe via the capture sequence. The ligation product and/or the capture probe can be extended using each other as an extension template, thereby incorporating the spatial barcode of the capture probe, or a complement thereof, into the extended products. Optionally, the extended products can be amplified and further process (e.g., sequenced) to determine the location of the non-coding RNA in the biological sample.

In some examples, one or both probes include one or more locked nucleic acids as described herein to increase probe specificity for non-coding RNAs. The one or more locked nucleic acids can be in a region of the probes that hybridizes to a non-coding RNA. In some examples, a minor groove binder (e.g., any of the exemplary minor groove binders described herein) is added before, simultaneously, or after the probe pair is contacted with the biological sample. In some examples, one or both probes include one or more phosphorothioate bonds to protect the probe(s) from degradation.

FIG. 15 shows an embodiment where capture from the biological sample occurs on the capture slide (e.g., the biological sample is placed on the capture slide), however, it is appreciated that the biological sample can be on a different substrate and the probe pair, and optionally a minor groove binder are contacted with the biological sample, followed by bringing the substrate with the biological sample in a sandwich configuration as described herein with the array including the plurality of capture probes.

### Example 4. Spatial Capture of Non-Coding RNA with a Splint Oligonucleotide

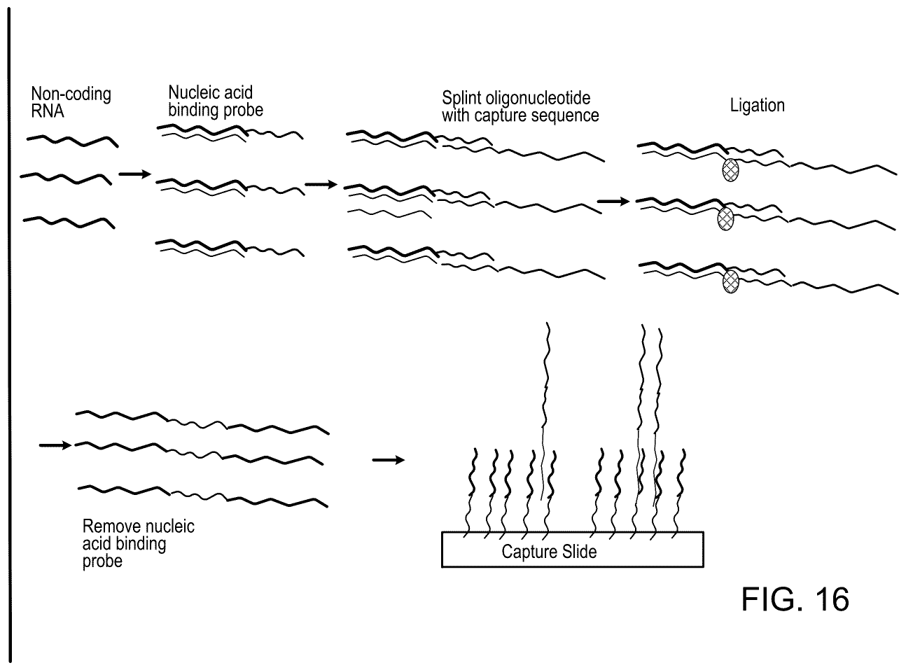

FIG. 16 is a schematic diagram showing an exemplary workflow for spatial capture of non-coding RNA via a splint oligonucleotide. More specifically, FIG. 16 shows non-coding RNAs (e.g., any of the non-coding RNAs described herein) present in a biological sample, followed by contacting the biological sample with a plurality of nucleic acid binding probes, where each nucleic acid binding probe includes a domain capable of hybridizing to the non-coding RNA and a ligation handle sequence that is substantially complementary to a portion splint oligonucleotide. Next, the biological sample is contacted with a plurality of splint oligonucleotides where a splint oligonucleotide includes a substantially complementary sequence to the ligation handle of the nucleic acid binding probe and a capture sequence that is capable of interacting (e.g., hybridizing) with a capture domain of a capture probe. Once hybridized to the nucleic acid binding probe, the splint oligonucleotide is ligated to the non-coding RNA to generate a ligation product, and the DNA binding probe is removed from the ligation product. In some examples, the nucleic acid probe is removed (e.g., melted) through the use of temperature, ionic strength, and probe length as described herein. The ligation product (e.g., ligated non-coding RNA and splint oligonucleotide) interacts (e.g., hybridizes) with the capture domain of a capture probe on an array. The ligation product and/or the capture probe can be extended using each other as extension templates, thereby incorporating the spatial barcode of the capture probe, or a complement thereof, into the extended products. See, Chapin, S C. et al., Angew Chem Int Ed Engl., 50 (10): 2289-93 (2011), which is hereby incorporated by reference, for describing a ligation based approach for miRNA detection.

FIG. 16 shows an embodiment where capture from the biological sample is performed in situ e.g., that the biological sample can be on a different substrate and the nucleic acid binding probe, the splint oligonucleotide, and optionally one or more minor groove binders are contacted with the biological sample followed by bringing the substrate with the biological sample in a sandwich configuration as described herein with the array including the plurality of capture probes, however, it is appreciated that capture from the biological sample can occur on the capture slide (e.g., the biological sample is placed on the capture slide).

### EMBODIMENTS

Embodiment 1 is a method of determining a location of a non-coding RNA in a biological sample, the method comprising: (a) providing an array comprising a plurality of capture probes, wherein a capture probe of the plurality of capture probes comprises: (i) a spatial barcode and (ii) a capture domain; (b) contacting the biological sample with a probe, wherein the probe comprises: (i) a ncRNA-binding domain comprising a nucleic acid sequence capable of hybridizing to at least a portion of the non-coding RNA and (ii) a nucleic acid capture sequence capable of hybridizing to at least a portion of the capture domain; (c) hybridizing the ncRNA-binding domain of (i) the probe to the non-coding RNA; (d) hybridizing the nucleic acid capture sequence of (ii) the probe to the capture domain of the capture probe; and (e) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

Embodiment 2 is the method of embodiment 1, wherein the non-coding RNA is a siRNA.

Embodiment 3 is the method of embodiment 1, wherein the non-coding RNA is a miRNA.

Embodiment 4 is the method of embodiment 3, wherein the miRNA is a pri-miRNA.

Embodiment 5 is the method of embodiment 3, wherein the miRNA is a pre-miRNA.

Embodiment 6 is the method of any one of embodiments 1-5, wherein the probe comprises one or more locked nucleic acids, optionally wherein the ncRNA-binding domain comprises the one or more locked nucleic acids.

Embodiment 7 is the method of any one of embodiments 1-6, wherein before or during step (c), the method comprises the use of one or more minor groove binders.

Embodiment 8 is the method of embodiment 7, wherein the one or more minor groove binders comprises one or more of duocarmycin A, Chromomycin A3, and alkamin.

Embodiment 9 is the method of any one of embodiments 1-8, wherein the biological sample is disposed on the array.

Embodiment 10 the method of any one of embodiments 1-8, wherein the biological sample is disposed on a first substrate.

Embodiment 11 is the method of embodiment 10, wherein the method further comprises aligning the substrate with the array, such that at least a portion of the biological sample is aligned with at least a portion of the array, optionally wherein the array is comprised on a second substrate.

Embodiment 12 is the method of any one of embodiments 1-11, wherein the method further comprises releasing the non-coding RNA from the probe after step (c).

Embodiment 13 is the method of embodiment 12, wherein the releasing comprises the use of an RNase, a protease, or a combination thereof.

Embodiment 14 is the method of embodiment 13, wherein the RNase is one or more of RNase A, RNase P, RNase T1, or RNase H, optionally wherein the protease is Proteinase K.

Embodiment 15 is the method of any one of embodiments 1-14, wherein the probe further comprises a nucleic acid sequence comprising a non-coding RNA barcode disposed between the ncRNA-binding domain of the probe and the nucleic acid capture sequence of the probe.

Embodiment 16 is the method of embodiment 15, wherein the nucleic acid sequence of the non-coding RNA barcode identifies the non-coding RNA hybridized to the probe.

Embodiment 17 is the method of any one of embodiments 1-16, wherein the method further comprises extending the capture probe using the probe as an extension template after step (d), thereby generating an extended capture probe.

Embodiment 18 is the method of embodiment 17, wherein the extending comprises the use of a polymerase.

Embodiment 19 is the method of any one of embodiments 1-18, wherein the method further comprises the use of a template switch oligonucleotide after step (d).

Embodiment 20 is the method of any one of embodiments 17-19, wherein the method further comprises extending the probe using the capture probe as a template after step (d), thereby generating a sequence complementary to the extended capture probe.

Embodiment 21 is a method of determining a location of a non-coding RNA in a biological sample, the method comprising: (a) providing an array comprising a plurality of capture probes, wherein a capture probe of the plurality of capture probes comprises: (i) a spatial barcode and (ii) a capture domain; (b) contacting the biological sample with a probe, wherein the probe comprises a ncRNA-binding domain comprising a nucleic acid sequence capable of hybridizing to at least a portion of the non-coding RNA; (c) hybridizing the probe to the non-coding RNA and extending the probe using the non-coding RNA as an extension template, thereby generating an extended probe; (d) incorporating a polynucleotide sequence comprising at least three nucleotides to the 3′ end of the extended probe; (e) hybridizing an adaptor to the polynucleotide sequence, wherein the adaptor comprises: (i) a nucleic acid sequence that hybridizes to the polynucleotide sequence and (ii) a nucleic acid sequence complementary to a nucleic acid capture sequence, wherein the nucleic acid capture sequence is capable of hybridizing to at least a portion of the capture domain; (f) extending the extended probe using the adaptor as an extension template, thereby incorporating the nucleic acid capture sequence into the extended probe; (g) hybridizing the nucleic acid capture sequence of the extended probe to the capture domain; and (h) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

Embodiment 22 is the method of embodiment 21, wherein the non-coding RNA is a siRNA.

Embodiment 23 is the method of embodiment 21, wherein the non-coding RNA is a miRNA.

Embodiment 24 is the method of embodiment 22, wherein the miRNA is a pri-miRNA.

Embodiment 25 is the method of embodiment 22, wherein the miRNA is a pre-miRNA.

Embodiment 26 is the method of any one of embodiments 21-25, wherein the probe comprises one or more locked nucleic acids, optionally wherein the ncRNA-binding domain comprises the one or more locked nucleic acids.

Embodiment 27 is the method of any one of embodiments 21-26, wherein before or during step (c), the method further comprises using one or more minor groove binders.

Embodiment 28 is the method of embodiment 27, wherein the one or more minor groove binders comprises one or more of duocarmycin A, Chromomycin A3, and alkamin.

Embodiment 29 is the method of any one of embodiments 21-28, wherein the biological sample is disposed on the array.

Embodiment 30 is the method of any one of embodiments 21-28, wherein the biologicals sample is disposed on a first substrate, optionally the array is disposed on a second substrate.

Embodiment 31 is the method of embodiment 30, wherein the method further comprises aligning the first substrate with the array, such that at least a portion of the biological sample is aligned with at least a portion of the array.

Embodiment 32 is the method of any one of embodiments 21-30, wherein the method further comprises after step (f) releasing the non-coding RNA from the extended probe.

Embodiment 33 is the method of embodiment 32, wherein the releasing comprises the use of an exonuclease and/or a protease.

Embodiment 34 is the method of embodiment 33, wherein the exonuclease is a T7 exonuclease and/or the protease is Proteinase K.

Embodiment 35 is the method of any one of embodiments 21-33, wherein the adaptor comprises a template switch oligonucleotide.

Embodiment 36 is the method of embodiment 35, wherein 5′ end of the template switch oligonucleotide is phosphorylated.

Embodiment 37 is the method of any one of embodiments 21-33, 35, and 36 wherein the exonuclease is lambda exonuclease.

Embodiment 38 is the method of any one of embodiments 21-37, wherein the method further comprises after step (g), extending the capture probe using the extended probe as a template, thereby generating an extended capture probe.

Embodiment 39 is the method of embodiment 38, wherein the extending comprises the use of a polymerase.

Embodiment 40 is the method of embodiment 39, wherein the polymerase is a DNA polymerase.

Embodiment 41 the method of any one of embodiments 38-40, wherein the method further comprises generating a second strand complementary to the extended capture probe.

Embodiment 42 is the method of any one of embodiments 21-41, wherein the probe comprises one or more locked nucleic acids, optionally wherein the ncRNA-binding domain comprises the one or more locked nucleic acids.

Embodiment 43 is the method of any one of embodiments 21-42, wherein the polynucleotide sequence is a heteropolynucleotide sequence.

Embodiment 44 is the method of any one of embodiments 21-42, wherein the polynucleotide sequence is a homopolynucleotide sequence.

Embodiment 45 is a method of determining a location of a non-coding RNA in a biological sample, the method comprising: (a) providing an array comprising a plurality of capture probes, wherein a capture probe of the plurality of capture probes comprises: (i) a spatial barcode and (ii) a capture domain; (b) contacting the biological sample with a plurality of first probes and a plurality of second probes, wherein the plurality of first probes and the plurality of second probes target a plurality of non-coding RNAs in the biological sample, and wherein a first probe of the plurality of first probes and a second probe of the plurality of second probes comprise sequences that are capable of hybridizing to the non-coding RNA, and wherein the second probe comprises a nucleic acid capture sequence that is capable of hybridizing to at least a portion of the capture domain of the capture probe; (c) hybridizing the first probe and the second probe to the non-coding RNA; (d) ligating the first probe and the second probe, thereby generating a ligation product; (e) hybridizing the nucleic acid capture sequence of the ligation product to the capture domain of the capture probe; and (f) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

Embodiment 46 is the method of embodiment 45, wherein the non-coding RNA is a siRNA.

Embodiment 47 is the method of embodiment 45, wherein the non-coding RNA is a miRNA.

Embodiment 48 is the method of embodiment 47, wherein the miRNA is a pri-miRNA.

Embodiment 49 is the method of embodiment 47, wherein the miRNA is a pre-miRNA.

Embodiment 50 is the method of any one of embodiments 45-49, wherein the first probe and/or the second probe comprises one or more locked nucleic acids.

Embodiment 51 is the method of any one of embodiments 45-50, wherein before or during step (c), the method further comprises the use of one or more minor groove binders.

Embodiment 52 is the method of embodiment 51, wherein the one or more minor groove binders comprises one or more of duocarmycin A, Chromomycin A3, and alkamin.

Embodiment 53 is the method of any one of embodiments 45-52, wherein the biological sample is disposed on the array.

Embodiment 54 is the method of any one of embodiments 45-52, wherein the biological sample is disposed on a first substrate.

Embodiment 55 is the method of embodiment 54, wherein the method further comprises aligning the first substrate with the array, such that at least a portion of the biological sample is aligned with at least a portion of the array, optionally wherein the array is disposed on a second substrate.

Embodiment 56 is the method of any one of embodiments 45-55, wherein the method further comprises after step (d) releasing the non-coding RNA from the ligation product.

Embodiment 57 is the method of embodiment 56, wherein the releasing comprises the use of an RNase, a protease, or a combination thereof.

Embodiment 58 is the method of embodiment 57, wherein the RNase is one or more of RNase A, RNase P, RNase T1, or RNase H, optionally wherein the protease is Proteinase K.

Embodiment 59 is the method of any one of embodiments 45-58, wherein the nucleic acid capture sequence comprises a homopolymeric sequence or a fixed sequence.

Embodiment 60 is the method of embodiment 59, wherein the homopolymeric sequence is a poly(A) sequence.

Embodiment 61 is the method of any one of embodiments 45-60, wherein the method further comprises extending the capture probe using the ligation product as a template, thereby generating an extended capture probe.

Embodiment 62 is the method of embodiment 61, wherein the extending comprises the use of a polymerase.

Embodiment 63 is the method of embodiment 61 or 62, wherein the method further comprises extending the ligation product using the extended capture probe as a template, thereby generating an extended ligation product complementary to the extended capture probe.

Embodiment 64 is the method of any one of embodiments 45-63, wherein the first probe and/or the second probe comprises one or more locked nucleic acids.

Embodiment 65 is a method of determining a location of a non-coding RNA in a biological sample, the method comprising: (a) providing an array comprising a plurality of capture probes, wherein a capture probe of the plurality of capture probes comprises: (i) a spatial barcode and (ii) a capture domain; (b) contacting the biological sample with a plurality of probes, wherein a probe of the plurality of probes comprises: (i) a nucleic acid sequence capable of hybridizing to at least a portion of the non-coding RNA and (ii) a ligation handle; (c) contacting the biological sample with a plurality of splint oligonucleotides, wherein a splint oligonucleotide of the plurality of splint oligonucleotides comprises: (i) a sequence capable of hybridizing to at least a portion of the ligation handle and (ii) a nucleic acid capture sequence capable of hybridizing to at least a portion of the capture domain of the capture probe; (d) hybridizing the probe to the non-coding RNA and the splint oligonucleotide; (e) ligating the non-coding RNA to the splint oligonucleotide, thereby generating a ligation product; (f) hybridizing the nucleic acid capture sequence of the ligation product to the capture domain of the capture probe; and (g) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

Embodiment 66 is the method of embodiment 65, wherein the non-coding RNA is a siRNA.

Embodiment 67 is the method of embodiment 65, wherein the non-coding RNA is a miRNA.

Embodiment 68 is the method of embodiment 67, wherein the miRNA is a pri-miRNA.

Embodiment 69 is the method of embodiment 67, wherein the miRNA is a pre-miRNA.

Embodiment 70 is the method of any one of embodiments 65-69, wherein the probe and/or the splint oligonucleotide comprises one or more locked nucleic acids.

Embodiment 71 is the method of any one of embodiments 65-70, wherein the method further comprises, before or during step (d) the use of one or more minor groove binders.

Embodiment 72 is the method of embodiment 71, wherein the one or more minor groove binders comprises one or more of duocarmycin A, Chromomycin A3, and alkamin.

Embodiment 73 is the method of any one of embodiments 65-72, wherein the biological sample is disposed on the array.

Embodiment 74 is the method of any one of embodiments 65-72, wherein the biological sample is disposed on a first substrate.

Embodiment 75 is the method of embodiment 74, wherein the method further comprises aligning the first substrate with the array such that at least a portion of the biological sample is aligned with at least a portion of the array, optionally wherein the array is disposed on a second substrate.

Embodiment 76 is the method of any one of embodiments 65-75, wherein the method further comprises releasing the probe from the ligation product.

Embodiment 77 is the method of embodiment 76, wherein the releasing comprises the use of heat.

Embodiment 78 is the method of any one of embodiments 65-77, wherein the method further comprises extending the capture probe using the ligation product as a template, thereby generating an extended capture probe.

Embodiment 79 is the method of embodiment 78, wherein the extending comprises the use of a polymerase.

Embodiment 80 is the method of embodiment 78 or 79, wherein the method further comprises extending the ligation product, thereby generating an extended ligation product complementary to the extended capture probe.

Embodiment 81 is the method of any one of embodiments 65-80, wherein the nucleic acid capture sequence of the splint oligonucleotide comprises a homopolymeric sequence or a fixed sequence.

Embodiment 82 is the method of embodiment 81, wherein the homopolymeric sequence is a poly(A) sequence.

Embodiment 83 is the method of any one of embodiments 1-81, wherein the method further comprises, before step (c), the use of one or more Argonaute proteins.

Embodiment 84 is the method of embodiment 83, wherein the one or more Argonaute proteins comprises one or more of: AGO1, AGO2, AGO3, and AGO4.

Embodiment 85 is the method of any one of embodiments 1-84, wherein the method further comprises imaging the biological sample.

Embodiment 86 is the method of any one of embodiments 1-85, wherein the method further comprises staining the biological sample.

Embodiment 87 is the method of embodiment 86, wherein the staining comprises hematoxylin and/or eosin staining.

Embodiment 88 is the method of embodiment 86, wherein the staining comprises the use of a detectable label selected from the group consisting of a radioisotope, a fluorophore, a chemiluminescent compound, a bioluminescent compound, or a combination thereof.

Embodiment 89 is the method of any one of embodiments 1-88, wherein the capture probe comprises a cleavage domain, one or more functional domains, a unique molecular identifier, or combinations thereof.

Embodiment 90 is the method of any one of embodiments 1-89, wherein the capture domain of the capture probe comprises a homopolymeric sequence.

Embodiment 91 is the method of embodiment 90, wherein the homopolymeric sequence is a poly(T) sequence.

Embodiment 92 is the method of any one of embodiments 1-89, wherein the capture domain of the capture probe comprises a fixed sequence.

Embodiment 93 is the method of any one of embodiments 1-92, wherein the method further comprises permeabilizing the biological sample.

Embodiment 94 is the method of embodiment 93, wherein the permeabilizing comprises the use of a protease.

Embodiment 95 is the method of embodiment 94, wherein the protease comprises pepsin or proteinase K.

Embodiment 96 is the method of any one of embodiments 1-95, wherein the array comprises one or more features selected from the group consisting of: a spot, an inkjet spot, a masked spot, a pit, a post, a well, a ridge, a divot, a hydrogel pad, and a bead.

Embodiment 97 is the method of any one of embodiments 1-20, wherein the method further comprises migrating the probe to the array before step (d).

Embodiment 98 is the method of any one of embodiments 21-44, further comprising migrating the extended probe of step (f) to the array.

Embodiment 99 is the method of any one of embodiments 45-82, further comprising migrating the ligation product to the array.

Embodiment 100 is the method of any one of embodiments 97-99, wherein the migrating comprises electrophoresis.

Embodiment 101 is the method of any one of embodiments 1-100, wherein the biological sample is a tissue sample.

Embodiment 102 is the method of embodiment 101, wherein the tissue sample is a fresh-frozen tissue sample.

Embodiment 103 is the method of embodiment 102, wherein the tissue sample is a fixed tissue sample, and optionally, wherein the fixed tissue sample is a formalin-fixed paraffin-embedded tissue sample, an acetone-fixed tissue sample, a methanol-fixed tissue sample, or a paraformaldehyde-fixed tissue sample.

Embodiment 104 is the method of any one of embodiments 1-100, wherein the biological sample is a tissue section.

Embodiment 105 is the method of embodiment 104, wherein the tissue section is a fresh-frozen tissue section.

Embodiment 106 is the method of embodiment 104, wherein the tissue section is a fixed tissue section.

Embodiment 107 is the method of embodiment 106, wherein the fixed tissue section is a formalin-fixed paraffin-embedded tissue section, an acetone-fixed tissue section, a methanol-fixed tissue section, or a paraformaldehyde-fixed tissue section.

Embodiment 108 is a kit comprising: (a) an array comprising a plurality of capture probes, wherein a capture probe of the plurality of capture probes comprises: (i) a spatial barcode and (ii) a capture domain; and (b) a plurality of probes, wherein a probe of the plurality of probes comprises (i) a nucleic acid sequence complementary to a non-coding RNA and (ii) a nucleic acid capture sequence capable of hybridizing to at least a portion of the capture domain.

Embodiment 109 is the kit of embodiment 108, wherein the kit further comprises a reverse transcriptase.

Embodiment 110 is the kit of embodiment 108 or 109, wherein the kit further comprises a DNA polymerase.

Embodiment 111 is the kit of any one of embodiments 108-110, wherein the kit further comprises one or more minor groove binders.

Embodiment 112 is the kit of embodiment 111, wherein the one or more minor groove binders comprises one or more of duocarmycin A, Chromomycin A3, and alkamin.

Embodiment 113 is the kit of any one of embodiments 108-112, wherein the kit further comprises one or more permeabilization reagents.

Embodiment 114 is the kit of embodiment 113, wherein the one or more permeabilization reagents comprises one or more of a protease, a lipase, an RNase, a detergent, and a surfactant.

Embodiment 115 is a kit comprising: (a) an array comprising a plurality of capture probes, wherein a capture probe of the plurality of capture probes comprises: (i) a spatial barcode and (ii) a capture domain; (b) a plurality of probes, wherein a probe of the plurality of probes comprises a nucleic acid sequence capable of hybridizing to at least a portion of a non-coding RNA; (c) a reverse transcriptase; and (d) a plurality of adaptors, wherein an adaptor of the plurality of adaptors comprises: (i) a nucleic acid sequence capable of hybridizing to a polynucleotide sequence comprising at least three nucleotides added to an extended version of the probe and (ii) a nucleic acid sequence complementary to a nucleic acid capture sequence, wherein the nucleic acid capture sequence is capable of hybridizing to at least a portion of the capture domain of the capture probe.

Embodiment 116 is the kit of embodiment 115, wherein the kit further comprises a DNA polymerase.

Embodiment 117 is the kit of embodiment 115 or 116, wherein the kit further comprises one or more minor groove binders.

Embodiment 118 is the kit of embodiment 117, wherein the one or more minor grove binders comprises one or more of duocarmycin A, Chromomycin A3, and alkamin.

Embodiment 119 is the kit of any one of embodiments 115-118, wherein the kit further comprises one or more permeabilization reagents.

Embodiment 120 is the kit of embodiment 119, wherein the one or more permeabilization reagents comprises one or more of a protease, a lipase, an RNase, a detergent, and a surfactant.

Embodiment 121 is the kit of any one of embodiments 115-120, wherein the kit further comprises a plurality of dNTPs.

Embodiment 122 is the kit of embodiment 121, wherein the plurality of dNTPs comprises a plurality of dCTPs.

Embodiment 123 a kit comprising: (a) an array comprising a plurality of capture probes, wherein a capture probe of the plurality of capture probes comprises: (i) a spatial barcode and (ii) a capture domain; and (b) a plurality of first probes and a plurality of second probes, wherein a first probe of the plurality of first probes and a second probe of the plurality of second probes comprise sequences that are capable of hybridizing to a non-coding RNA, and herein the second probe comprises a nucleic acid capture sequence that is capable of hybridizing to at least a portion of the capture domain of the capture probe.

Embodiment 124 is the kit of embodiment 123, wherein the kit further comprises a ligase.

Embodiment 125 is the kit of embodiment 123 or 124, wherein the kit further comprises a polymerase.

Embodiment 126 is the kit of embodiment 125, wherein the polymerase is a DNA polymerase.

Embodiment 127 is the kit of any one of embodiments 123-126, wherein the kit further comprises one or more minor groove binders.

Embodiment 128 is the kit of embodiment 127, wherein the one or more minor grove binders comprises one or more of duocarmycin A, Chromomycin A3, and alkamin.

Embodiment 129 is the kit of any one of embodiments 123-128, wherein the kit further comprises one or more permeabilization reagents.

Embodiment 130 is the kit of embodiment 129, wherein the one or more permeabilization reagents comprises one or more of a protease, a lipase, an RNase, a detergent, and a surfactant.

Embodiment 131 is a kit comprising: (a) an array comprising a plurality of capture probes, wherein a capture probe of the plurality of capture probes comprises: (i) a spatial barcode and (ii) a capture domain; (b) a plurality of probes, wherein a probe of the plurality of probes comprises: (i) a nucleic acid sequence capable of hybridizing to at least a portion of a non-coding RNA and (ii) a ligation handle; and (c) a plurality of splint oligonucleotides, wherein a splint oligonucleotide of the plurality of splint oligonucleotides comprises: (i) a sequence capable of hybridizing to at least a portion of the ligation handle and (ii) a nucleic acid capture sequence capable of hybridizing to at least a portion of the capture domain of the capture probe.

Embodiment 132 is the kit of embodiment 131, wherein the kit further comprises a ligase.

Embodiment 133 is the kit of embodiment 131 or 132, wherein the kit further comprises a polymerase.

Embodiment 134 is the kit of embodiment 133, wherein the polymerase is a DNA polymerase.

Embodiment 135 is the kit of any one of embodiments 131-134, wherein the kit further comprises one or more minor groove binders.

Embodiment 136 is the kit of embodiment 135, wherein the one or more minor grove binders comprises one or more of duocarmycin A, Chromomycin A3, and alkamin.

Embodiment 137 is the kit of any one of embodiments 131-136, wherein the kit further comprises one or more permeabilization reagents.

Embodiment 138 is the kit of embodiment 137, wherein the one or more permeabilization reagents comprises one or more of a protease, a lipase, an RNase, a detergent, and a surfactant.

Embodiment 139 is the kit of any one of embodiments 108-138, wherein the kit further comprises one or more Argonaute proteins.

Embodiment 140 is the kit of embodiment 139, wherein the one or more Argonaute proteins comprises one or more of: AGO1, AGO2, AGO3, and AGO4.

Embodiment 141 is a composition comprising: (a) an array comprising a plurality of capture probes, wherein a capture probe of the plurality of capture probes comprises: (i) a spatial barcode and (ii) a capture domain; and (b) a probe hybridized to a non-coding RNA, wherein the probe comprises: (i) a nucleic acid sequence hybridized to at least a portion of the non-coding RNA and (ii) a nucleic acid capture sequence capable of hybridizing to at least a portion of the capture domain of the capture probe.

Embodiment 142 is the composition of embodiment 141, wherein the composition further comprises a reverse transcriptase.

Embodiment 143 is the composition of embodiment 141 or 142, wherein the composition further comprises a polymerase.

Embodiment 144 is the composition of any one of embodiments 141-143, wherein the probe comprises one or more locked nucleic acids.

Embodiment 145 is the composition of any one of embodiments 141-144, wherein the composition further comprises one or more minor grove binders.

Embodiment 146 is the composition of embodiment 145, wherein the one or more minor groove binders comprises one or more of duocarmycin A, Chromomycin A3, and alkamin.

Embodiment 147 is the composition of any one of embodiments 141-146, wherein the composition further comprises one or more Argonaute proteins.

Embodiment 148 is the composition of embodiment 147, wherein the one or more Argonaute proteins comprises one or more of AGO1, AGO2, AGO3, and AGO4.

Embodiment 149 is the composition of any one of embodiments 141-148, wherein the composition further comprises one or more permeabilization reagents comprising one or more of a protease, a lipase, an RNase, a detergent, and a surfactant.

Embodiment 150 is the composition of any one of embodiments 141-149, wherein the nucleic acid capture sequence is hybridized to at least a portion of the capture domain of the capture probe.

Embodiment 151 is the composition of embodiment 150, wherein the capture probe has been extended by using the probe as a template.

Embodiment 152 is the composition of embodiment 150 or 151, wherein the probe has been extended by using the capture probe as a template.

Embodiment 153 is a composition comprising: (a) an array comprising a plurality of capture probes, wherein a capture probe of the plurality of capture probes comprises: (i) a spatial barcode and (ii) a capture domain; (b) a plurality of probes, wherein a probe of the plurality of probes comprises a nucleic acid sequence capable of hybridizing to at least a portion of a non-coding RNA; (c) a reverse transcriptase; and (d) a plurality of adaptors, wherein an adaptor of the plurality of adaptors comprises: (i) a nucleic acid sequence capable of hybridizing to a polynucleotide sequence comprising at least three nucleotides added to an extended version of the probe and (ii) a nucleic acid sequence complementary to a nucleic acid capture sequence, wherein the nucleic acid capture sequence is capable of hybridizing to at least a portion of the capture domain.

Embodiment 154 is the composition of embodiment 153, wherein the composition further comprises a DNA polymerase.

Embodiment 155 is the composition of embodiment 153 or 154, wherein the composition further comprises one or more minor groove binders.

Embodiment 156 is the composition of embodiment 155, wherein the one or more minor groove binders comprises one or more of duocarmycin A, Chromomycin A3, and alkamin.

Embodiment 157 is the composition of any one of embodiments 153-156, wherein the composition further comprises one or more permeabilization reagents.

Embodiment 158 is the composition of embodiment 157, wherein the one or more permeabilization reagents comprises one or more of a protease, a lipase, an RNase, a detergent, and a surfactant.

Embodiment 159 is the composition of any one of embodiments 153-158, wherein the composition further comprises one or more Argonaute proteins.

Embodiment 160 is the composition of embodiment 159, wherein the one or more Argonaute proteins comprises one or more of AGO1, AGO2, AGO3, and AGO4.

Embodiment 161 is the composition of any one of embodiments 153-160, wherein the probe has been extended using the non-coding RNA as an extension template to generate the extended version of the probe.

Embodiment 162 is the composition of embodiment 161, wherein the extended probe further comprises the polynucleotide sequence at its 3′ end.

Embodiment 163 is the composition of embodiment 162, wherein the nucleic acid sequence (i) of the adaptor is hybridized to the polynucleotide sequence.

Embodiment 164 is the composition of embodiment 163, wherein the extended probe has been further extended using the adaptor as an extension template, thereby incorporating the nucleic acid capture sequence into the extended probe.

Embodiment 165 is the composition of embodiment 164, wherein the nucleic acid capture sequence of the extended probe is hybridized to at least a portion of the capture domain of the capture probe.

Embodiment 166 is the composition of embodiment 165, wherein the capture probe has been extended using the extended probe as an extension template.

Embodiment 167 is the composition of embodiment 164 or 165, wherein the extended probe has been extended using the capture probe as an extension template.

Embodiment 168 is a composition comprising: (a) an array comprising a plurality of capture probes, wherein a capture probe of the plurality of capture probes comprises: (i) a spatial barcode and (ii) a capture domain; (b) a plurality of first probes, wherein a first probe of the plurality of first probes comprises a sequence that is capable of hybridizing to at least a portion of a non-coding RNA; (c) a plurality of second probes, wherein a second probe of the plurality of second probes comprises: (i) a sequence that is capable of hybridizing to at least a portion of the non-coding RNA and (ii) a nucleic acid capture sequence that is capable of hybridizing to at least a portion of the capture domain of the capture probe; and (d) a ligase.

Embodiment 169 is the composition of embodiment 168, wherein the composition further comprises a polymerase.

Embodiment 170 is the composition of embodiment 168 or 169, wherein the composition further comprises one or more minor groove binders.

Embodiment 171 is the composition of embodiment 170, wherein the one or more minor groove binders comprises one or more of duocarmycin A, Chromomycin A3, and alkamin.

Embodiment 172 is the composition of any one of embodiments 1685-171, wherein the composition further comprises one or more permeabilization reagents.

Embodiment 173 is the composition of embodiment 172, wherein the one or more permeabilization reagents comprises one or more of a protease, a lipase, an RNase, a detergent, and a surfactant.

Embodiment 174 is the composition of any one of embodiments 168-173, wherein the composition further comprises one or more Argonaute proteins.

Embodiment 175 is the composition of embodiment 174, wherein the one or more Argonaute proteins comprises one or more of AGO1, AGO2, AGO3, and AGO4.

Embodiment 176 is the composition of any one of embodiments 168-175, wherein the first probe and the second probe have been ligated to generate a ligation product.

Embodiment 177 is the composition of embodiment 176, wherein the nucleic acid capture sequence of the ligation product is hybridized to at least a portion of the capture domain of the capture probe.

Embodiment 178 is the composition of embodiment 177, wherein the capture probe has been extended using the ligation product as a template.

Embodiment 179 is the composition of embodiment 176 or 177, wherein the ligation product has been extended using the capture probe as a template.

Embodiment 180 is a composition comprising: (a) an array comprising a plurality of capture probes, wherein a capture probe of the plurality of capture probes comprises: (i) a spatial barcode and (ii) a capture domain; (b) a plurality of probes, wherein a probe of the plurality of probes comprises: (i) a nucleic acid sequence capable of hybridizing to at least a portion of a non-coding RNA and (ii) a ligation handle; and (c) a plurality of splint oligonucleotides, wherein a splint oligonucleotide of the plurality of splint oligonucleotides comprises: (i) a sequence capable of hybridizing to at least a portion of the ligation handle and (ii) a nucleic acid capture sequence capable of hybridizing to at least a portion of the capture domain of the capture probe.

Embodiment 181 is the composition of embodiment 180, wherein the composition further comprises a ligase.

Embodiment 182 is the composition of embodiment 180 or 181, wherein the composition further comprises a polymerase.

Embodiment 183 is the composition of any one of embodiments 180-182, wherein the probe comprises one or more locked nucleic acids.

Embodiment 184 is the composition of any one of embodiments 180-183, wherein the composition further comprises one or more minor grove binders.

Embodiment 185 is the composition of embodiment 184, wherein the one or more minor groove binders comprises one or more of duocarmycin A, Chromomycin A3, and alkamin. Embodiment 186 is the composition of any one of embodiments 180-185, wherein the composition further comprises one or more Argonaute proteins.

Embodiment 187 is the composition of embodiment 186, wherein the one or more Argonaute proteins comprises one or more of AGO1, AGO2, AGO3, and AGO4.

Embodiment 188 is the composition of any one of embodiments 180-187, wherein the composition further comprises one or more permeabilization reagents comprising one or more of a protease, a lipase, an RNase, a detergent, and a surfactant.

Embodiment 189 is the composition of any one of embodiments 180-188, wherein the non-coding RNA and the splint oligonucleotide have been ligated, thereby generating a ligation product.

Embodiment 190 is the composition of embodiment 189, wherein the nucleic acid capture sequence of the ligation product is hybridized to the capture domain of the capture probe.

Embodiment 191 is the composition of embodiment 190, wherein the capture probe has been extended using the ligation product as an extension template.

Embodiment 192 is the composition of embodiment 190 or 191, wherein the ligation product has been extended using the capture probe as an extension template.

Embodiment 193 is the composition of any one of embodiments 141-190, wherein the non-coding RNA is a siRNA.

Embodiment 194 is the composition of any one of embodiments 141-190, wherein the non-coding RNA is a miRNA.

Embodiment 195 is the composition of embodiment 194, wherein the miRNA is a pri-miRNA.

Embodiment 196 is the composition of embodiment 194, wherein the miRNA is a pre-miRNA.

Embodiment 197 is the composition of any one of embodiments 141-196, wherein the capture sequence comprises a homopolymeric sequence.

Embodiment 198 is the composition of embodiment 197, wherein the homopolymeric sequence comprises a poly(A) sequence.

Embodiment 199 is the composition of any one of embodiments 141-198, wherein the nucleic acid capture sequence comprises a fixed sequence.

Embodiment 200 is the composition of any one of embodiments 141-199, wherein the capture domain comprises a poly(T) sequence.

Embodiment 201 is the composition of any one of embodiments 141-200, wherein the capture domain comprises a fixed sequence.

Embodiment 202 is the composition of any one of embodiments 141-201, where the array comprises one or more features selected from the group consisting of: a spot, an inkjet spot, a masked spot, a pit, a post, a well, a ridge, a divot, a hydrogel pad, and a bead.

Embodiment 203 is the composition of any one of embodiments 141-201, wherein the composition further comprises a biological sample.

Embodiment 204 is the composition of embodiment 203, wherein the biological sample is a tissue section.

Embodiment 205 is the composition of embodiment 204, wherein the tissue section is a fresh-frozen tissue section.

Embodiment 206 is the composition of embodiment 204, wherein the tissue section is a fixed tissue section.

Embodiment 207 is the composition of embodiment 206, wherein the fixed tissue section is a formalin-fixed paraffin-embedded tissue section, an acetone-fixed tissue section, a methanol-fixed tissue section, or a paraformaldehyde-fixed tissue section.

Embodiment 208 is the composition of embodiment 203, wherein the biological sample is a tissue sample.

Embodiment 209 is the composition of embodiment 208, wherein the tissue sample is a fixed tissue sample.

What is claimed is:

1. A method of determining a location of a non-coding RNA in a biological sample, the method comprising:
(a) providing an array comprising a plurality of capture probes, wherein a capture probe of the plurality of capture probes comprises: (i) a spatial barcode and (ii) a capture domain;
(b) contacting the biological sample with a plurality of probes, wherein a probe of the plurality of probes comprises: (i) a nucleic acid sequence capable of hybridizing to at least a portion of the non-coding RNA and (ii) a ligation handle;
(c) contacting the biological sample with a plurality of splint oligonucleotides, wherein a splint oligonucleotide of the plurality of splint oligonucleotides comprises: (i) a sequence capable of hybridizing to at least a portion of the ligation handle and (ii) a nucleic acid capture sequence capable of hybridizing to at least a portion of the capture domain of the capture probe;
(d) hybridizing the probe to the non-coding RNA and the splint oligonucleotide;
(e) ligating the non-coding RNA to the splint oligonucleotide, thereby generating a ligation product;
(f) hybridizing the nucleic acid capture sequence of the ligation product to the capture domain of the capture probe; and
(g) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the biological sample.

2. The method of claim 1, wherein step (d) occurs prior to step (c) and/or after step (b).

3. The method of claim 1, wherein the non-coding RNA is a siRNA or miRNA, optionally wherein the miRNA is a pri-miRNA or a pre-miRNA.

4. The method of claim 1, wherein the probe and/or the splint oligonucleotide comprises one or more locked nucleic acids.

5. The method of claim 1, wherein the method further comprises, before or during step (d) the use of one or more minor groove binders, optionally wherein the one or more minor groove binders comprises one or more of duocarmycin A, Chromomycin A3, and alkamin.

6. The method of claim 1, wherein the biological sample is disposed on the array.

7. The method of claim 1, wherein the biological sample is disposed on a first substrate and the array is disposed on a second substrate.

8. The method of claim 7, wherein the method further comprises aligning the first substrate with the second substrate such that at least a portion of the biological sample is aligned with at least a portion of the array.

9. The method of claim 1, wherein the method further comprises releasing or separating the probe from the ligation product, optionally wherein the releasing comprises the use of heat.

10. The method of claim 1, wherein the method further comprises extending the capture probe using the ligation product as a template, thereby generating an extended capture probe comprising a sequence complementary to the ligation product; and/or extending the ligation product using the capture probe as a template, thereby generating an extended ligation product comprising a sequence complementary to the capture probe.

11. The method of claim 1, wherein the nucleic acid capture sequence of the splint oligonucleotide comprises a homopolymeric sequence or a fixed sequence.

12. The method of claim 11, wherein the homopolymeric sequence is a poly(A) sequence.

13. The method of claim 1, wherein the non-coding RNA is complexed with one or more Argonaute proteins, wherein the one or more Argonaute proteins comprises one or more of: AGO1, AGO2, AGO3, and AGO4, optionally wherein the one or more Argonaute proteins lacks cleavage activity.

14. The method of claim 1, wherein the method further comprises imaging the biological sample.

15. The method of claim 1, wherein the method further comprises staining the biological sample, wherein the staining comprises: (i) hematoxylin and/or eosin staining or (ii) a radioisotope, a fluorophore, a chemiluminescent compound, a bioluminescent compound, or a combination thereof.

16. The method of claim 1, wherein the capture probe comprises a cleavage domain, one or more functional domains, a unique molecular identifier, or a combination thereof.

17. The method of claim 1, wherein the capture domain of the capture probe comprises a fixed sequence or a homopolymeric sequence, optionally wherein the homopolymeric sequence is a poly(T) sequence.

18. The method of claim 1, wherein the method further comprises permeabilizing the biological sample, wherein the permeabilizing comprises the use of a protease, optionally wherein the protease comprises pepsin or proteinase K.

19. The method of claim 1, wherein the biological sample is a tissue section, optionally a fresh frozen tissue section or a fixed frozen tissue section.

20. The method of claim 1, wherein the method further comprises migrating the ligation product to the array or migrating the capture probe from the array to the biological sample, optionally wherein the capture probe comprises a cleavage domain and the method further comprises cleaving the capture probe at the cleavage domain to release the capture probe from the array.

21. The method of claim 10, wherein the determining comprises sequencing; optionally wherein the method further comprises sequencing the extended capture probe or a complement thereof or sequencing the extended ligation or a complement thereof.

22. A method of determining a location of a non-coding RNA in a tissue sample, the method comprising:
(a) providing an array comprising a plurality of capture probes, wherein a capture probe of the plurality of capture probes comprises: (i) a spatial barcode and (ii) a capture domain;
(b) contacting the tissue sample with a plurality of probes, wherein a probe of the plurality of probes comprises: (i) a nucleic acid sequence capable of hybridizing to at least a portion of the non-coding RNA and (ii) a ligation handle;
(c) hybridizing the probe to the non-coding RNA, thereby generating a probe: non-coding RNA complex;
(d) contacting the tissue sample with a plurality of splint oligonucleotides, wherein a splint oligonucleotide of the plurality of splint oligonucleotides comprises: (i) a sequence capable of hybridizing to the ligation handle and (ii) a nucleic acid capture sequence capable of hybridizing to at least a portion of the capture domain of the capture probe;
(e) hybridizing the splint oligonucleotide to the probe: non-coding RNA complex, wherein the sequence capable of hybridizing to the ligation handle of the splint oligonucleotide hybridizes to the ligation handle of the probe in the probe: non-coding RNA complex;
(f) coupling the non-coding RNA to the splint oligonucleotide, thereby generating a connected probe;
(g) hybridizing the connected probe to the capture probe, optionally comprising hybridizing the nucleic acid capture sequence of the connected probe to the capture domain of the capture probe; and
(h) determining (i) all or a portion of the sequence of the non-coding RNA, or a complement thereof, and (ii) the sequence of the spatial barcode, or a complement thereof, and using the determined sequences of (i) and (ii) to determine the location of the non-coding RNA in the tissue sample.

23. The method of claim 22, wherein the coupling in step (f) comprises ligation, optionally wherein the ligation comprises chemical ligation or enzymatic ligation, optionally wherein the enzymatic ligation comprises use of a T4 RNA ligase (Rnl2), a PBCV-1 DNA Ligase or Chlorella virus DNA Ligase, a single-stranded DNA ligase, or a T4 DNA ligase.

24. The method of claim 22, further comprising after step (f), releasing or separating the probe from the connected probe, optionally wherein the releasing or separating comprises the use of heat.

25. The method of claim 22, further comprising extending the capture probe using the connected probe as a template, thereby generating an extended capture probe comprising a sequence complementary to the connected probe; and/or extending the connected probe using the capture probe as a template, thereby generating an extended connected probe comprising a sequence complementary to the capture probe.

26. The method of claim 25, wherein the determining comprises sequencing; optionally wherein the method further comprises sequencing the extended capture probe or a complement thereof or sequencing the extended connected probe or a complement thereof.

27. The method of claim 23, wherein a 3′ end of the non-coding RNA is ligated to a 5′ end of the splint oligonucleotide.

28. The method of claim 22, wherein the non-coding RNA is a siRNA or a miRNA, optionally wherein the miRNA is a pri-miRNA or a pre-miRNA.

29. The method of claim 22, wherein the method further comprises migrating the connected probe to the array or migrating the capture probe from the array to the biological sample, optionally wherein the capture probe comprises a cleavage domain and the method further comprises cleaving the capture probe at the cleavage domain to release the capture probe from the array.

30. The method of claim 22, wherein the biological sample is a tissue section, optionally a fresh frozen tissue section or a fixed frozen tissue section.
